(19) United States
(12) Patent Application Publication
(10) Pub. No.: US 2026/0334818 A1
(43) Pub. Date: Oct. 29, 2026

(54) SYSTEM AND METHOD FOR ASSESSING MOVEMENT ACTIVITY IN INFANTS

(71) Applicant: AngelEye Health, Nashville, TN (US)

(72) Inventors: Marissa KOSCIELSKI, Cincinnati, OH (US); Patrick TINSLEY, Hoboken, NJ (US); Adrian RODRIGUEZ, Temple City, CA (US); David LANDMAN, Nashville, TN (US); Christopher RAND, Brentwood, TN (US)

(21) Appl. No.: 19/660,054

(22) Filed: Apr. 27, 2026

Related U.S. Application Data
(60) Provisional application No. 63/794,580, filed on Apr. 25, 2025.
(60) Provisional application No. 63/794,986, filed on Apr. 25, 2025.

(51) Int. Cl. A61B 5/11; A61B 5/00

(52) U.S. Cl. CPC A61B 5/1123; A61B 5/7275; A61B 2503/06

(57) ABSTRACT

A system and method for detecting, assessing and evaluating movement activity of an infant using sensor‑based data. The sensor data associated with the infant is preprocessed and analyzed to determine spatial representations of anatomical landmarks over time. Pose data is generated from the spatial representations and is subjected to data cleaning and normalization to produce normalized pose data. Temporal changes in the normalized pose data are processed to identify and characterize infant movement activity. The system generates movement feature data comprising structured representations of the movement activity, enabling objective assessment of infant motion based on pose‑derived information.

### RELATED APPLICATIONS

The present application claims priority to U.S. provisional patent application Serial No. 63/794,580, filed on Apr. 25, 2025, and entitled SYSTEM AND METHOD FOR DETECTING ASYMMETRICAL MOVEMENT IN NEONATES, and claims priority to U.S. provisional patent application Serial No. 63/794,986, filed on Apr. 25, 2025, and entitled SYSTEM AND METHOD FOR DETECTING ASYMMETRICAL MOVEMENT IN NEONATES, the contents of which are herein incorporated by reference.

### BACKGROUND OF THE INVENTION

In modern hospital environments, particularly in neonatal intensive care units (NICUs) and pediatric intensive care units (PICUs), continuous physiological monitoring of infants is a standard and essential component of medical care. Infants, including neonates and post-neonatal infants, often require close observation due to their physiological vulnerability, underdeveloped organ systems, and increased risk of sudden adverse events. As such, conventional infant monitoring systems are employed to track selected biometric or biological parameters of the infants to ensure early detection of medical complications and to support timely clinical intervention.

Traditional infant monitoring systems typically include a combination of sensors and devices configured to monitor selected parameters, such as heart rate, respiratory rate, oxygen saturation (SpO2), blood pressure, and body temperature. Electrocardiogram (ECG) leads are frequently used for cardiac monitoring, while pulse oximeters are employed to assess blood oxygen levels and perfusion. Respiratory effort is often monitored via impedance pneumography or respiratory inductance plethysmography, and skin-contact thermistors or axillary probes are used for continuous temperature monitoring.

One specific example of conventional infant monitoring includes systems designed to reduce the risk of Sudden Infant Death Syndrome (SIDS). These conventional systems typically track breathing patterns and movement, triggering alerts in the event of prolonged apnea (cessation of breathing), abnormal heart rates (bradycardia or tachycardia), or low oxygen saturation. While such systems have been helpful in clinical settings, they are often reactive rather than predictive and may suffer from high false alarm rates or limited adaptability to individual patient baselines.

Despite advances in medical technology, conventional infant monitoring systems often involve bulky equipment, numerous wired connections, and limited integration of data sources. These limitations often impede clinical workflow, increase caregiver burden, and reduce infant comfort.

### SUMMARY OF THE INVENTION

The present invention is directed to systems and methods for monitoring, detecting, and assessing infants, including neonates and post-neonatal infants, within a healthcare facility, including for example Neonatal Intensive Care Units (NICUs) and Pediatric Intensive Care Units (PICUs). The system provides a non-invasive, sensor-integrated platform configured to capture, process, and analyze multimodal data representative of infant movement and physiological state to generate objective, quantitative assessments of infant health and development.

The advanced infant detection and assessment system can be configured to employ one or more sensing modalities, including non-contact imaging devices such as cameras and, in certain embodiments, contact-based sensors, such as wearable or bed-integrated sensing elements, to acquire data streams associated with infant motion, posture, and other types of physiological conditions. The acquired sensor-based data streams can be processed using one or more computational models, including computer vision techniques and machine learning models, to transform the raw, unstructured sensor data into structured representations of movement, behavior, and physiological features. The detection and assessment system can be configured to generate quantitative movement-based metrics and derived assessment outputs that characterize neuromotor function and other clinically relevant conditions. The metrics can include, for example, measures of movement symmetry or asymmetry, limb coordination, activity levels, posture, and temporal motion dynamics. In addition, the system can analyze gaze patterns, spinal alignment, anti-gravity strength, and other functional indicators associated with infant development. By processing temporal sequences of captured data, the detection and assessment system can identify trends, detect anomalies, and recognize motion signatures corresponding to physiological or neurological conditions, including seizure activity, systemic distress, or developmental irregularities.

In certain embodiments, the detection and assessment system can be configured to further integrate and correlate movement-derived metrics with physiological data obtained from additional sensors to provide a unified, multimodal assessment of infant health. The system can thus be configured to combine heterogeneous data sources to generate composite scores, condition likelihood indicators, and predictive assessments that reflect individualized infant baselines and evolving clinical states.

The detection and assessment system can also be configured to capture image and/or video data of an infant and to process the captured data through a suitable data processing pipeline or technique that performs feature extraction and motion tracking. The processed data can be analyzed using one or more computational models to generate quantitative assessments representative of infant movement and condition. In certain embodiments, additional sensing components, such as inertial measurement units (IMUs), pressure sensors, or environmental sensors, can be employed to provide supplemental data streams that enhance robustness, accuracy, and contextual understanding of the assessment of the infant related data. The system can be further configured to present real-time assessment results, longitudinal trends, and alert conditions to clinicians in formats suitable for integration into clinical workflows.

The detection and assessment system can be further configured for detecting, quantifying, and analyzing infant movement to generate objective movement quantification outputs, including movement symmetry or asymmetry measures, coordination metrics, and activity-pattern indicators. The quantified movement metrics can assist clinicians in the early detection of acute and chronic neurological or orthopedic conditions in the infants, enable tracking of developmental progression, and support the personalization of intervention strategies. In certain embodiments, the quantified movement data is further utilized to perform point-of-care assessments of neuromotor function and neuromuscular development, as well as to evaluate additional functional and physiological indicators, including gaze patterns, spinal curvature, and anti-gravity strength. The system can also analyze motion and behavioral patterns associated with physiological states or conditions, including seizures, sepsis, and jaundice, by identifying characteristic temporal or spatial signatures within the captured data.

Advantageously, the present invention provides a scalable and extensible platform for continuous, non-invasive monitoring and objective assessment of infant health and development. By transforming multimodal sensor data into quantitative metrics and clinically interpretable insights, the system enables improved detection, monitoring, and evaluation of neuromotor and physiological conditions and supports informed clinical decision-making in neonatal and pediatric care environments.

The present invention is also directed another embodiment of the infant detection and assessment system configured to provide non-invasive evaluation of infant (e.g., neonatal) health and development in a healthcare facility. The detection and assessment system can employ one or more non-contact sensing modalities to capture sensor data associated with the infant while reducing stress and discomfort and enabling point-of-care assessment. The system can be configured to process multimodal sensor data to generate indicators, classifications, and/or scores associated with multiple assessment domains, including neuromotor characteristics, neuromuscular development, gaze-related behavior, posture-related characteristics (e.g., spinal alignment or curvature), anti-gravity movement, and physiological states. In certain embodiments, the system can convert the sensor data into movement-related data by identifying and characterizing motion of the infant based on spatial and temporal analysis of pose and sensor-derived inputs. The movement-related data can be categorized into one or more movement classifications representing movement types, states, or patterns, and, in some embodiments, can be further processed to generate one or more scores or indices associated with the infant’s movement or physiological characteristics.

In certain embodiments, the detection and assessment system can employ one or more computational models to determine spatial characteristics of the infant’s body and to process pose data and associated sensor-derived inputs to generate structured, quantitative or semi-quantitative outputs, including classification data, health-related scores, and associated confidence values. The system can optionally integrate additional sensors, including thermal sensors, physiological sensors, or other sensing modalities, to augment captured data. The system can further be configured to present the generated outputs via one or more display interfaces. In this manner, the system transforms multimodal sensor data into structured, machine-interpretable representations, including movement data, categorized outputs, and optional scores, that support monitoring and analysis of infant health and development based on the captured data.

The present invention is also directed to a detection and assessment system configured to identify patterns associated with selected infection-related conditions, including sepsis, based on an analysis of multimodal data obtained from an infant in a healthcare facility. The system can include one or more sensors configured to capture sensor data associated with the infant, and a plurality of processing units configured to extract and analyze movement-related data, skin-related optical feature data, and physiological data. A vitals extraction unit can generate physiological data representing one or more physiological parameters, including heart rate, respiratory rate, oxygenation, or perfusion-related indicators, while a skin tone extraction unit can generate skin-related optical feature data representing observable characteristics such as color variation or perfusion-related features. A classification unit can be configured to analyze the multimodal data to identify co-occurring patterns, including variations in physiological data, changes in skin-related optical features, and changes in movement characteristics, and to categorize such patterns as being associated with physiological instability or infection-related conditions. In certain embodiments, an optional health scoring unit can generate one or more scores or indices representing the degree or trend associated with the identified patterns. A display unit can present the classifications, scores, and associated trends, and can generate alerts when predefined conditions or multimodal pattern combinations are satisfied, thereby facilitating identification of patterns associated with sepsis and enabling timely clinical evaluation.

In certain embodiments, the present invention is directed to a detection and assessment system configured to identify patterns associated with jaundice based on analysis of multimodal data, including skin-related optical feature data derived from sensor data associated with an infant. The system can include a skin tone extraction unit configured to process image, video, and/or optical sensor data to extract skin-related optical features, including color-based features, chromaticity information, and normalized color metrics indicative of changes in skin coloration. The skin tone extraction unit can analyze one or more regions of interest corresponding to exposed skin areas, including facial regions, torso regions, or extremities, and can track such features over time to identify temporal trends or progressive changes in skin coloration. A classification unit can categorize the extracted features into classifications associated with normal coloration or color variation patterns indicative of jaundice, and can analyze temporal changes to identify persistent or progressive color shifts. In certain embodiments, the system can optionally employ multimodal analysis by combining the skin-related optical feature data with movement-related data and physiological data to improve robustness. An optional health scoring unit can generate scores representing the degree or progression of jaundice-associated patterns, and a display unit can present the classifications, scores, and trends, and can generate alerts when predefined conditions associated with jaundice-related patterns are satisfied, thereby enabling non-invasive identification of patterns associated with jaundice.

The present invention is directed to a detection and assessment system for assessing movement activity of an infant. The system comprises one or more sensors configured to generate sensor data associated with the infant; a data acquisition unit configured to receive the sensor data and to generate processed sensor data by performing one or more preprocessing operations on the sensor data; a pose estimation unit configured to process the processed sensor data to determine spatial representations of anatomical landmarks of the infant over time and to generate pose data representing the spatial representations of the anatomical landmarks over time; a data cleaning and normalization unit configured to process the pose data by applying one or more data cleaning and normalization operations and to generate normalized pose data representing the spatial representations of the anatomical landmarks over time; and a movement extraction unit configured to process the normalized pose data representing the spatial representations of the anatomical landmarks over time to identify and characterize the movement activity based on temporal changes in the spatial representations, and to generate movement feature data having structured representations of the movement activity.

The system can also include at least one comparator unit configured to receive the movement feature data and to generate comparison data associated with the movement activity, where the comparison data includes at least one of limb movement comparison data representing intra-limb movement activity and inter-limb comparison data representing movement activity across contralateral anatomical regions. The system can further include a movement variance quantification unit configured to receive at least one of the limb movement comparison data or the inter-limb comparison data and to generate movement variability data representing variability or asymmetry of the movement activity. Still further, the system can include a display unit configured to receive at least one of the movement feature data and the comparison data and to display representations of the movement activity, including at least one of movement feature representations, comparative representations, or the movement variability data to the user.

According to one practice, the system can include a limb movement comparator unit configured to receive and process the movement feature data and to perform a temporal comparison of movement activity of a selected limb based on the movement feature data, and to generate limb movement comparison data including structured representations of temporal changes in the movement activity for the selected limb; a cross-midline movement comparator unit configured to receive and process the movement feature data and to perform a comparison of movement activity between contralateral anatomical regions of the infant derived from the spatial representations of the anatomical landmarks based on the movement feature data, and to generate inter-limb comparison data including structured representations of comparative relationships between the movement activity of the contralateral anatomical regions; and a movement variance quantification unit configured to (i) receive the limb movement comparison data to generate movement variability data representing variability of the movement activity of the selected limb, (ii) receive the inter-limb comparison data to generate movement variability data representing asymmetry or coordination of the movement activity across the contralateral anatomical regions, or (iii) receive both the limb movement comparison data and the inter-limb comparison data to generate movement variability data representing integrated variability and asymmetry of the movement activity, including relationships between variability of the selected limb and asymmetry across the contralateral anatomical regions. Still further, the system includes a display unit configured to receive at least one of the limb movement comparison data or the inter-limb comparison data and to present representations of the movement activity, including representations of temporal changes in movement activity for the selected limb and representations of comparative relationships between movement activity of the contralateral anatomical regions.

According to one practice, the limb movement comparator unit can be configured to perform a temporal comparison of movement activity of a selected limb based on the movement feature data across temporal intervals to detect changes, trends, or deviations in the movement activity. The cross-midline movement comparator unit can be configured to perform a comparison of movement activity between contralateral anatomical regions of the infant based on the movement feature data and to generate inter-limb comparison data comprising structured representations of comparative relationships between the movement activity of the contralateral anatomical regions, including relative differences, timing relationships, or coordination.

The movement variance quantification unit can be configured to process (i) the limb movement comparison data independently to generate intra-limb variability metrics, (ii) the inter-limb comparison data independently to generate asymmetry metrics associated with contralateral anatomical regions, or (iii) both the limb movement comparison data and the inter-limb comparison data to generate integrated variability and asymmetry metrics. The movement feature data can include trajectories of anatomical landmarks and associated movement features including at least one of displacement, velocity, acceleration, frequency, duration, or range of motion. The movement activity comprises at least one of discrete movement events, continuous movement states, or temporal movement patterns characterizing the movement of the infant over time.

The display unit can be configured to generate one or more alerts based on one or more predefined conditions associated with at least one of the movement activity, variability of the movement activity, or asymmetry of the movement activity. The movement variance quantification unit can be configured to determine temporal trends in variability or asymmetry of the movement activity over one or more time intervals, and the limb movement comparator unit can be configured to perform the temporal comparison across one or more temporal windows including sliding or overlapping intervals. Further, the movement extraction unit is configured to identify and classify the movement activity into at least one of discrete movement events, continuous movement states, or temporal movement patterns, and the movement variance quantification unit is configured to determine relationships between intra-limb variability and inter-limb asymmetry of the movement activity. The alerts are configured to prompt further evaluation.

The present invention is also directed to a computer-implemented method for assessing movement activity of an infant, comprising receiving sensor data associated with the infant from one or more sensors; generating processed sensor data by performing one or more preprocessing operations on the sensor data; processing the processed sensor data to determine spatial representations of anatomical landmarks of the infant over time and generating pose data representing the spatial representations of the anatomical landmarks over time; processing the pose data by applying one or more data cleaning and normalization operations to generate normalized pose data representing the spatial representations of the anatomical landmarks over time; and processing the normalized pose data representing the spatial representations of the anatomical landmarks over time to identify and characterize movement activity based on temporal changes in the spatial representations, and generating movement feature data comprising structured representations of the movement activity.

According to one practice, the method of the invention further includes receiving the movement feature data and generating comparison data associated with the movement activity, the comparison data including at least one of (i) limb movement comparison data representing intra-limb movement activity or (ii) inter-limb comparison data representing movement activity across contralateral anatomical regions. The method can also include receiving at least one of the limb movement comparison data or the inter-limb comparison data and generating movement variability data representing variability or asymmetry of the movement activity. The methods can also include presenting representations of the movement activity, including at least one of movement feature representations, comparative representations, or movement variability data.

According to another practice, the method further includes performing a temporal comparison of movement activity of a selected limb based on the movement feature data and generating limb movement comparison data comprising structured representations of temporal changes in the movement activity for the selected limb; performing a comparison of movement activity between contralateral anatomical regions of the infant derived from the spatial representations of the anatomical landmarks based on the movement feature data and generating inter-limb comparison data including structured representations of comparative relationships between the movement activity of the contralateral anatomical regions; and (i) processing the limb movement comparison data to generate movement variability data representing variability of the movement activity of the selected limb, (ii) processing the inter-limb comparison data to generate movement variability data representing asymmetry or coordination of the movement activity across the contralateral anatomical regions, or (iii) processing both the limb movement comparison data and the inter-limb comparison data to generate movement variability data representing integrated variability and asymmetry of the movement activity, including relationships between variability of the selected limb and asymmetry across the contralateral anatomical regions.

The method can also include performing the temporal comparison across one or more temporal intervals to detect changes, trends, or deviations in the movement activity, and generating inter-limb comparison data including relative differences, timing relationships, or coordination between the contralateral anatomical regions. Still further, the method includes (i) processing the limb movement comparison data independently to generate intra-limb variability metrics, (ii) processing the inter-limb comparison data independently to generate asymmetry metrics associated with contralateral anatomical regions, or (iii) processing both the limb movement comparison data and the inter-limb comparison data to generate integrated variability and asymmetry metrics.

The movement feature data can include trajectories of the anatomical landmarks over time and one or more associated movement features including at least one of displacement, velocity, acceleration, frequency, duration, or range of motion. The movement activity can include at least one of discrete movement events, continuous movement states, or temporal movement patterns characterizing movement of the infant over time.

The method still further includes generating one or more alerts based on one or more predefined conditions associated with at least one of the movement activity, variability of the movement activity, or asymmetry of the movement activity; determining temporal trends in variability or asymmetry of the movement activity over one or more time intervals; identifying and classifying the movement activity into at least one of discrete movement events, continuous movement states, or temporal movement patterns; determining relationships between intra-limb variability and inter-limb asymmetry of the movement activity; and/or generating the one or more alerts to prompt further evaluation.

### BRIEF DESCRIPTION OF THE DRAWING

These and other features and advantages of the present invention will be more fully understood by reference to the following detailed description in conjunction with the attached drawings in which like reference numerals refer to like elements throughout the different views. The drawings illustrate principals of the invention and, although not to scale, show relative dimensions.

FIG. 1 is a schematic block diagram of a detection system according to the teachings of the present invention.

FIG. 2 is a schematic block diagram of another embodiment of a detection and assessment system according to the teachings of the present invention.

FIG. 3 is a schematic block diagram of yet another embodiment of a detection and assessment system according to the teachings of the present invention for determining and assessing sepsis and jaundice in an infant.

### DETAILED DESCRIPTION OF THE INVENTION

The present invention is directed to systems and methods for non-invasive detection, monitoring, and assessment of infant health and development using multimodal data derived from one or more sensing modalities. In certain embodiments, the detection and assessment system can include one or more sensors configured to capture sensor data associated with an infant, such as a neonate, including image data, video data, thermal data, and/or other physiological or movement-related data. The detection and assessment system can further include one or more processing units configured to transform the sensor data into structured data representations, including movement-related data, skin-related optical feature data, and physiological data. The processing can include, for example, pose estimation to determine spatial characteristics of the infant, extraction of skin-related optical features to identify observable characteristics associated with physiological states, and extraction of physiological parameters, including vital signs, from contact-based and/or non-contact sensing modalities.

The detection and assessment system can further include one or more data processing and analysis units configured to normalize, align, and integrate the multimodal data, and to analyze the data to generate categorized or classified representations, scores, and/or other outputs associated with the infant’s condition. In certain embodiments, a classification unit can identify patterns in the multimodal data, including co-occurring variations across movement-related data, skin-related optical feature data, and physiological data, and can categorize such patterns as being associated with selected physiological conditions. An optional scoring unit can generate quantitative representations, including indices or trends associated with the identified patterns. A display unit can present the generated data in one or more user interfaces and can generate alerts when predefined conditions or pattern combinations are satisfied. In this manner, the system can transform heterogeneous sensor-derived data into structured, interpretable outputs that facilitate non-invasive, real-time or near real-time assessment of infant health.

In certain embodiments, a first implementation of the system can be configured to assess infant movement and motor development based primarily on movement-related data derived from pose estimation and motion analysis. In other embodiments, the detection and assessment system can be configured to perform multimodal physiological assessment by integrating movement-related data with skin-related optical feature data and physiological data to identify patterns associated with physiological conditions, including infection-related conditions such as sepsis. In further embodiments, the system can be configured to identify patterns associated with jaundice based on analysis of skin-related optical features, including temporal changes in skin coloration, optionally in combination with other data types to improve robustness. These and other embodiments provide flexible, extensible frameworks for multimodal assessment of infant health using non-invasive sensing and data-driven analysis.

As used herein, the term “artificial intelligence” or “AI” refers to any computational technique configured to perform tasks that typically require human intelligence, including tasks involving perception, recognition, classification, prediction, reasoning, recommendation, or decision-making, and including but not limited to machine learning employing machine learning models, deep learning, neural networks, computer vision, multimodal models, probabilistic models, rule-based systems, hybrid systems, and combinations thereof.

As used herein, the term “artificial intelligence system” or “AI system” refers to any computing system, software, hardware, firmware, or combination thereof configured to perform tasks that typically require human intelligence by receiving input data and generating one or more outputs based on learned patterns, programmed logic, probabilistic inference, or combinations thereof. The artificial intelligence system can process inputs to perform perception, recognition, classification, prediction, reasoning, recommendation, decision-making, generation, interpretation, or other cognitive or analytical operations. The artificial intelligence system can include, in whole or in part, one or more machine learning models, including generative models and generative language models, and can optionally further include rule-based logic, retrieval systems, knowledge bases, symbolic reasoning components, optimization engines, or other computational modules. In some embodiments, the artificial intelligence system can include large language models implemented as generative language models configured to produce natural language outputs, explanations, or structured responses. The artificial intelligence system can also include computer vision models, multimodal models, natural language processing systems, recommendation engines, ranking systems, probabilistic inference engines, reinforcement learning systems, hybrid symbolic-statistical systems, or combinations thereof. The artificial intelligence system can include, for example, AI assistants, conversational agents, recommendation systems, computer vision systems, multimodal systems, and other systems configured to process input data and generate outputs using one or more machine learning models.

The artificial intelligence system can operate using structured data, unstructured data, semi-structured data, images, audio, video, text, sensor data, contextual data, environmental data, multimodal inputs, or any combination thereof. The artificial intelligence system can generate outputs including classifications, predictions, recommendations, rankings, decisions, control signals, generated content, summaries, explanations, or other inferred results. The artificial intelligence system can be configured to perform tasks including, for example, object detection, optical character recognition, semantic understanding, natural language understanding, natural language generation, classification, clustering, regression, ranking, anomaly detection, feature extraction, representation learning, forecasting, planning, reasoning, and decision-making. In some embodiments, the artificial intelligence system can generate natural language outputs using one or more generative language models, including large language models. The artificial intelligence system can be trained using supervised learning, unsupervised learning, semi-supervised learning, self-supervised learning, reinforcement learning, transfer learning, federated learning, online learning, continual learning, or combinations thereof. The training can occur offline or online, and models within the artificial intelligence system can be updated periodically, continuously, or adaptively. The artificial intelligence system can be implemented in software, hardware accelerators, cloud-based platforms, edge devices, mobile devices, wearable devices, embedded systems, robotic systems, or distributed computing environments. The artificial intelligence system can include a single model or multiple models arranged in a pipeline, ensemble, cascade, or hierarchical architecture. The artificial intelligence system can further include preprocessing components, feature extraction modules, embedding generators, retrieval components, inference engines, scoring engines, ranking engines, orchestration modules, and output generation modules configured to operate in a coordinated manner.

As used herein, the term “machine learning model” or “model,” in either singular or plural form, refer to any computational, statistical, or mathematical model configured to learn patterns, relationships, features, or representations from data and to generate one or more outputs based on one or more inputs, without limitation. Such outputs may include, without limitation, predictions, classifications, scores, probabilities, embeddings, recommendations, rankings, transformations, generated content, decisions, control signals, or other inferred results. The machine learning model can represent a learned or configurable mapping, function, distribution, or decision boundary between input data and corresponding outputs, where parameters of the model are determined or configurable through a training process using data.

The machine learning models can be trained using labeled data, unlabeled data, partially labeled data, synthetic data, reinforcement signals, self-supervised objectives, or combinations thereof. The model training can occur offline, online, incrementally, periodically, continuously, adaptively, or using transfer learning from previously trained models. The training can involve optimization of parameters such as weights, coefficients, embeddings, or latent variables using gradient-based methods, probabilistic inference techniques, evolutionary algorithms, or other optimization approaches. Once trained, the machine learning model can be deployed for inference to generate outputs for previously unseen inputs.

The machine learning models can be configured to perform tasks including, without limitation, classification, regression, clustering, ranking, anomaly detection, recommendation, generation, transformation, dimensionality reduction, representation learning, sequence modeling, forecasting, optimization, control, or decision-making. The models can operate on any suitable data type, including text, images, audio, video, structured data, time-series data, sensor data, graph data, multimodal data, or combinations thereof, including sensor-derived data.

The machine learning models can include, for example, neural networks, deep neural networks, feedforward neural networks, convolutional neural networks (CNNs), recurrent neural networks (RNNs), long short-term memory networks (LSTMs), gated recurrent units (GRUs), transformer-based models, attention-based models, encoder-decoder architectures, autoencoders, variational autoencoders (VAEs), generative adversarial networks (GANs), diffusion models, graph neural networks (GNNs), multimodal models, and vision-language models. The machine learning models can also include classical or non-neural models, including decision trees, random forests, gradient boosted trees, boosting models, bagging models, support vector machines (SVMs), k-nearest neighbor models (KNN), naïve Bayes models, Bayesian networks, probabilistic graphical models, hidden Markov models, conditional random fields, linear regression models, logistic regression models, ridge regression models, lasso regression models, elastic net models, clustering models including k-means clustering, hierarchical clustering, density-based clustering, Gaussian mixture models, and dimensionality reduction models including principal component analysis (PCA), independent component analysis (ICA), and manifold learning techniques. The machine learning models can further include reinforcement learning models, such as Q-learning models, deep Q-networks (DQNs), policy gradient models, actor-critic models, model-based reinforcement learning systems, and multi-agent reinforcement learning models. The machine learning models can also include generative models, including generative language models, configured to generate new content based on learned data distributions. The machine learning models can be optionally combined into ensembles, cascaded pipelines, hierarchical systems, hybrid architectures, or multi-stage processing frameworks. The machine learning models can be implemented in software, hardware, firmware, or combinations thereof, and may be deployed in cloud computing environments, distributed systems, edge devices, mobile devices, or embedded systems.

As used herein, the term “generative language model”, in either singular or plural form, refers to a type of machine learning model configured to generate natural language content based on learned patterns, relationships, or representations derived from training data. The generative language model can receive one or more inputs, such as prompts, queries, instructions, contextual data, structured information, or multimodal inputs, and generate corresponding textual outputs including sentences, paragraphs, summaries, responses, translations, explanations, code, structured text, or other language-based content. The generative language models can be trained on large corpora of textual data using supervised learning, self-supervised learning, reinforcement learning, or combinations thereof, and can learn probabilistic relationships between tokens, words, phrases, semantic representations, or latent variables. The generative language models can generate text by, for example, predicting tokens sequentially, sampling from learned probability distributions, decoding latent representations, or otherwise producing language outputs conditioned on input context. The generated outputs can be deterministic or stochastic and can be controlled using decoding strategies, sampling parameters, constraints, or prompt conditioning.

The generative language models can include transformer-based architectures, autoregressive models, encoder-decoder models, sequence-to-sequence models, masked language models, diffusion-based language models, retrieval-augmented generation models, and hybrid symbolic-neural architectures. In many embodiments, generative language models can be implemented as large language models (LLMs) employing attention mechanisms and transformer architectures configured to model contextual dependencies across token sequences. Accordingly, large language models represent one implementation of generative language models, although generative language models can also include smaller-scale models or alternative architectures. The generative language models can be configured to perform tasks including, for example, text generation, dialogue generation, question answering, summarization, translation, instruction following, content drafting, classification with explanation, structured data generation, code generation, and multimodal text generation. In some embodiments, the generative language models can operate in conjunction with retrieval systems, knowledge bases, external tools, or additional machine learning models, and may be fine-tuned, instruction-tuned, domain-adapted, or otherwise specialized using additional training data, reinforcement learning, human feedback, or parameter-efficient adaptation techniques. The generative language model can form part of a broader generative artificial intelligence system configured to produce human-readable text and/or structured language outputs that are contextually relevant to provided inputs and can optionally operate in combination with models configured to process other modalities, including images, audio, video, or structured data, to generate multimodal language outputs.

In the present disclosure, data used to train a machine learning model can include data containing correlations that a machine learning process or technique may utilize to model relationships between two or more types or categories of data elements (“training data”). For example, and without limitation, the training data may comprise a plurality of data entries, each entry representing a set of data elements that were recorded, received, and/or generated together. The data elements may be correlated by shared co-occurrence within a data entry, proximity within the data, or other relationships. Multiple data entries within the training data may exhibit one or more trends or patterns in correlations between categories or types of data elements. For instance, and without limitation, a higher value of a first data element belonging to a first category or type of data element may tend to correlate with a higher value of a second data element belonging to a second category or type of data element, indicating a possible proportional or other mathematical relationship linking values across categories. Multiple categories of data elements may be related in the training data according to various correlations, which may indicate associative, predictive, and/or potentially causative relationships between categories of data elements. These correlations may be modeled as mathematical or statistical relationships by the machine learning processes described herein.

The training data may be formatted and/or organized by categories of data elements, for example by associating data elements with one or more descriptors corresponding to categories, although such categorization may be optional in some embodiments. The data elements can include, for example, image data, video data, frame sequences, and other file-based or sensor-derived data formats, including data generated by camera-based monitoring systems and corresponding extracted features. As a non-limiting example, training data may include data entered in standardized forms by persons or processes, such that entry of a given data element in a given field within a form may be mapped or correlated to one or more category descriptors. Elements in the training data may be linked to descriptors of categories or types by tags, tokens, or other data elements. For example, and without limitation, training data may be provided in fixed-length formats, formats linking positions of data to categories such as comma-separated value (CSV) formats, and/or self-describing formats such as extensible markup language (XML), or other structured or semi-structured formats, enabling processes or devices to detect categories of data.

Alternatively, or additionally, the training data may include one or more data elements that are not categorized, that is, the training data may not be formatted or contain descriptors for some elements of data. Machine learning models or algorithms and/or other processes may sort the training data according to one or more categorizations using, for instance, natural language processing algorithms, tokenization, detection of correlated values in raw data, and the like. The categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name or other types of data may be identified by reference to a list, dictionary, or other compendium of terms, permitting ad-hoc categorization by machine learning algorithms and/or automated association of data in the data entry with descriptors or into a given format. The ability to categorize data entries automatically may enable the same training data to be made applicable for two or more distinct machine learning algorithms as described in further detail below. Training data used by an electronic device may be used to correlate any input data as described in this disclosure to any output data as described in this disclosure.

As used herein, the term “artificial intelligence assistant” or “AI assistant” refers to any artificial intelligence system configured to receive one or more inputs and generate one or more outputs including responses, recommendations, analyses, predictions, classifications, rankings, decisions, actions, or other inferred results based at least in part on the received input and/or stored data, learned models, or contextual information. The AI assistant can include or utilize one or more machine learning models, including generative models and generative language models, and can optionally further include rule-based logic, retrieval systems, knowledge bases, optimization engines, or other computational components.

The AI assistant can be configured to interact with one or more users, systems, or environments, or to operate without direct user interaction, and may operate in conversational, command-driven, autonomous, or semi-autonomous modes. In some embodiments, the AI assistant can be implemented as a conversational agent configured to generate natural language responses using a generative language model. In other embodiments, the AI assistant can generate structured outputs, control signals, or recommendations without conversational interaction.

The AI assistant can receive inputs including, without limitation, text, speech, images, video, sensor data, location data, contextual data, structured data, user preference data, environmental data, or multimodal inputs. The AI assistant can generate outputs including responses, recommendations, rankings, classifications, explanations, comparisons, summaries, alerts, or action instructions. In some embodiments, the AI assistant can perform reasoning, retrieval, planning, decision-making, or workflow orchestration using one or more underlying models.

The AI assistant can include, for example, conversational agents, digital assistants, recommendation assistants, voice assistants, chatbots, multimodal assistants, robotic assistants, wearable device assistants, augmented reality assistants, virtual reality assistants, vehicle-based assistants, or autonomous agents. The AI assistant can operate on or in conjunction with mobile devices, wearable devices, computers, robotic systems, vehicles, kiosks, smart appliances, cloud platforms, edge devices, or distributed computing systems.

The AI assistant can further integrate multiple models, including one or more generative language models, computer vision models, recommendation models, ranking models, and decision models, which may operate in a coordinated pipeline, ensemble, or multi-stage architecture to produce outputs. The AI assistant can represent a type or implementation of an artificial intelligence system configured for assisting a user, system, or process.

As used herein, the term “data object” is intended to mean or refer to a structured unit of data that encapsulates one or more data attributes and is stored, processed, or transmitted within a system, such as a computing system or environment. The data object can be an instantiation of a corresponding data entity, wherein the data entity represents a conceptual model or schema defining the characteristics of similar data objects. Each data object can include metadata, relationships to other data objects, and specific values for attributes defined by the associated data entity. Data objects can exist in various forms, including database records, serialized data structures, or in-memory representations within software applications.

As used herein, the term “data entity” is intended to mean or refer to a distinct, identifiable unit of data or information that represents a real-world object, concept, or event within a data processing system. The data entity can be characterized by a unique identity and is associated with one or more data attributes that define the properties or characteristics of the data entity. The data entities serve as structured components within databases, information models, or data structures, facilitating organization, retrieval, and processing of related data.

As used herein, the term “attribute” or “data attribute” is generally intended to mean or refer to a characteristic, property, or descriptor associated with a data entity that describes an aspect of a data object or data entity. The data attributes can include a naming attribute, a descriptive attribute, and/or a referential attribute. The naming attribute can name an instance of a data object or entity. The descriptive attribute can be used to describe the characteristics or features or the relationship with the data object or entity. The referential attribute can be used to formalize binary and associative relationships and in referring to another instance of the attribute or data entity or data object stored at another location (e.g., in another table).

The term “application” or “software application” or “program” as used herein is intended to include or designate any type of procedural software application and associated software code which can be called or can call other such procedural calls or that can communicate with a user interface or access a data store. The software application can also include called functions, procedures, and/or methods.

As used herein, the term “electronic device” refers to any device or system including, for example, one or more processors, circuitry, memory devices, storage devices or systems, networking components, computers or computing systems, controllers, servers, mobile devices, tablets, smartphones, or combinations thereof, configured to execute instructions and/or perform operations. The electronic device can be implemented in cloud environments, servers, mobile devices, edge devices, embedded systems, or distributed computing systems. The electronic device can further include input/output interfaces, sensors, communication interfaces, or other hardware and software components.

As used herein, the term “neonates” means human infants from the time of birth through a selected neonatal period of life, typically including, for example, the first 28 days of life, independent of gestational age at delivery (e.g., including preterm, term, and post-term infants). In the context of the present invention, the neonates may be under continuous clinical care or monitoring in a healthcare (e.g., hospital) environment.

As used herein, the term “infant” means a human child from the time of birth up to 12 months of age. The term infant is intended to encompass both neonates and post-neonatal infants (e.g., from 29 days through 1 year). As such, this term describes patients within this age range who may require specialized medical care, monitoring, or intervention due to developmental, physiological, or health-related needs specific to this early stage of life. The term can also encompass infants born prematurely (preterm), at term, or post-term, irrespective of gestational age at delivery.

As used herein, the term “neonatal” refers to or is characteristic of the neonatal period and encompasses physiological, developmental, or clinical conditions associated with or occurring within neonates. The term includes infants born at full term, preterm (e.g., before 37 completed weeks of gestation), or post-term. Unless otherwise specified, references to “neonates” or “neonatal” in the present disclosure include all infants within this age range, regardless of health status or medical condition. In the context of in-hospital monitoring by associated monitoring systems, neonatal can encompass the monitoring and analysis of vital signs, physical parameters, and other clinical indicators pertinent to neonate health.

As used herein, the term “physiological data” means any measurable biological signal or parameter that reflects a functional state of a human subject, including an infant. The physiological data can include real-time or recorded information generated or derived from sensors, electrodes, or other monitoring devices that detect and quantify one or more biological or physical characteristics or parameters. The data can be used for diagnostic, therapeutic, detection, monitoring, or assessment purposes and can be acquired continuously or intermittently. The physiological data can include, for example, cardiovascular-related data, respiratory-related data, thermoregulatory-related data, neurological-related data, metabolic or chemical-related data, and body position, movement, or posture-related data. The physiological data can include vital sign-related features representing physiological measurements and sensor-derived features, such as skin-related optical features, that are indicative of physiological conditions”

As used herein, the term “healthcare facility” refers to any environment or setting in which medical care, monitoring, assessment, or treatment of a patient can be provided. A healthcare facility can include, for example, hospitals, neonatal intensive care units (NICUs), pediatric intensive care units (PICUs), clinics, outpatient care centers, rehabilitation facilities, long-term care facilities, and other clinical or care environments. In some embodiments, a healthcare facility can also include non-traditional or distributed care environments, such as home-based care settings, remote monitoring environments, or telemedicine-enabled care systems.

As used herein, the term “critical care personnel” refers to clinical care or healthcare providers, including professionals, caregivers, and support staff, who are trained, authorized, or otherwise qualified to provide direct or indirect medical or health care, monitoring, or support to patients within a healthcare facility, such as in an intensive or critical care setting. This includes, for example, settings such as a neonatal intensive care unit (NICU), a pediatric intensive care unit (PICU), and general intensive care units (ICUs). The term can include individuals involved in both clinical and operational roles associated with the assessment, treatment, stabilization, and support of critically ill patients, including neonates, infants, children, and adolescents. Examples of critical care personnel include physicians (e.g., neonatologists, pediatric intensivists, anesthesiologists), nurses (including registered nurses, nurse practitioners, and clinical nurse specialists with critical care training), respiratory therapists, clinical pharmacists, medical technicians and technologists, and therapists (such as physical, occupational, or speech therapists). The term can also include support staff such as unit clerks, care coordinators, medical social workers, and emergency transport personnel, including paramedics and flight nurses. This definition applies to both licensed medical practitioners and ancillary staff contributing to intensive care, whether in hospital-based, mobile, or telehealth-supported environments.

As used herein, the term “clinical care provider” refers to healthcare and support professionals who are trained, licensed, or otherwise qualified to deliver medical, therapeutic, diagnostic, or supportive services within a healthcare facility, such as a clinical setting or hospital, including, for example, critical care personnel. This term encompasses individuals engaged in the direct or indirect care of patients across various levels of acuity, including, for example, intensive care, acute care, emergency care, and specialty care environments. Clinical care providers can include, for example, physicians or doctors (e.g., intensivists, pediatricians, neonatologists, anesthesiologists), advanced practice providers such as nurse practitioners (NPs) and physician assistants (PAs), registered nurses (RNs), clinical nurse specialists (CNSs), and licensed practical nurses (LPNs). The providers can further include respiratory therapists, physical therapists, occupational therapists, speech-language pathologists, clinical pharmacists, diagnostic technologists (e.g., radiologic or ultrasound technologists), and laboratory personnel. Additionally, the providers can include care coordinators, case managers, medical social workers, and other allied health professionals involved in patient management, treatment planning, or clinical decision-making. This term is intended to cover individuals operating in hospital-based, outpatient, mobile, or telemedicine-enabled environments and may include both permanent and temporary staff, including those participating in multidisciplinary care teams.

As used herein, in the context of infant movement, the terms “asymmetry,” “asymmetrical,” or “asymmetrical movement” can refer to a lack of symmetry in movement activity, where symmetry corresponds to similarity or equivalence in movement characteristics across one or more anatomical regions of an infant. Asymmetry can therefore include differences in movement activity between two or more anatomical regions during voluntary or involuntary movement. The anatomical regions can include corresponding paired regions (e.g., left and right upper or lower limbs), as well as non-corresponding or non-paired regions (e.g., upper body relative to lower body, proximal versus distal regions, or localized regions relative to overall body movement). The differences can include, for example, differences in amplitude, frequency, velocity, range of motion, activity level, timing, coordination, or other kinematic or temporal characteristics of movement activity derived from spatial representations of anatomical landmarks.

In some embodiments, asymmetry can be determined based on analysis of movement activity derived from spatial representations of anatomical landmarks, including geometric, kinematic, temporal, or statistical analysis of pose data, movement feature data, or comparison data generated from one or more sensors. In certain embodiments, asymmetry can be quantified using one or more asymmetry measures, asymmetry metrics, or asymmetry indices, including measures of relative motion magnitude, activity distribution, timing differences, coordination differences, or regional movement imbalance across anatomical regions. The asymmetry measures can include, for example, comparisons of movement activity between contralateral regions, as well as comparisons between non-corresponding regions, including aggregated or composite measures such as gross body movement indices or comparisons between upper-body and lower-body movement activity. The quantified asymmetry measures can be used as indicators, or potential indicators, of neuromotor characteristics, developmental conditions, injury, or other physiological conditions in an infant.

As used herein, “multimodal data” refers to data derived from multiple sensing modalities and/or data types, including, for example, movement-related data, skin-related optical feature data, and physiological data, which may be obtained from one or more sensors.

As used herein, the term “movement activity” can refer to any detectable or characterizable motion-related behavior of a subject derived from temporal analysis of spatial representations of anatomical landmarks or body segments. The movement activity can include, for example, discrete movement events, continuous movement states, and temporal movement patterns. The discrete movement events can include temporally bounded movements having identifiable onset and offset, continuous movement states can include sustained conditions such as maintained posture or reduced motion, and temporal movement patterns can include sequences, repetitions, or coordinated movements occurring over time.

The present invention is directed to a detection and assessment system configured to process sensor data associated with an infant to generate structured representations of movement activity and to evaluate variability and asymmetry of such movement activity. The system includes a pose estimation unit that determines spatial representations of anatomical landmarks, a movement extraction unit that converts such spatial representations into movement feature data representing movement activity, and one or more comparator units that perform intra-limb and inter-limb analyses to generate comparison data. A movement variance quantification unit can optionally process the comparison data, independently or in combination, to generate variability and asymmetry metrics across anatomical regions. The system further includes a display unit configured to present movement activity, comparative analyses, and variability or asymmetry measures, and to generate alerts based on predefined conditions. In this manner, the system transforms sensor-derived data into structured, temporally derived representations of movement activity that enable real-time and longitudinal assessment of infant movement.

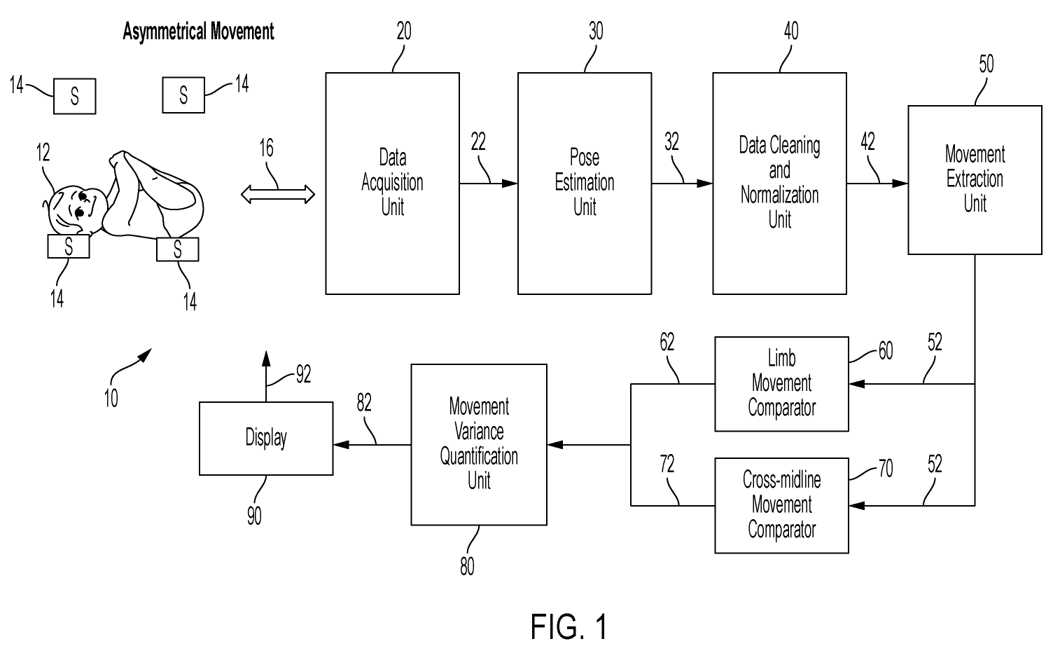

The present invention is directed a detection and assessment system for providing comprehensive, non-invasive evaluations of infant (e.g., neonatal) health and development in a healthcare facility. FIG. 1 is directed to one embodiment of the detection and assessment system 10 that can be configured for detecting, quantifying, and assessing movement asymmetry, movement variability, and related movement characteristics of body parts in infants, such as neonates. The illustrated detection and assessment system 10 can be configured to acquire, process, and analyze physiological data of an infant 12, such as a neonate, to generate structured representations of movement and derived quantitative movement assessments. In one embodiment, the infant detection and assessment system 10 can include one or more sensors 14 configured to monitor and detect selected types of physiological data of the infant 12 corresponding to movement, posture, or other types of physiological data representing physiological characteristics of the infant. Those of ordinary skill in the art will readily recognize that a single sensor or multiple sensors can be employed in the current system. For ease of description and for the sake of simplicity, the system is described as employing multiple sensors. The sensors 14 can be arranged at selected locations relative to the infant based on the type of sensors being employed. The sensors 14 can include, for example, contact (e.g., wearable) or non-contact sensors, such as for example accelerometers, pressure sensors, cameras, or other suitable types of sensors. The sensors 14 can be strategically placed on or about the infant 12 to capture selected types of physiological data, such as body movement data, inertial data, and physiological state data, such as heart rate, blood oxygen levels, respiratory rate, and the like. The sensors 14 can also include cameras to capture image and video data of the neonate, as well as thermal cameras to capture thermal data. The placement of the sensors 14 can be optimized to capture a comprehensive range of body movements. The sensors 14 can generate sensor data 16 indicative of the physiological data, such as movement data, that is received and processed by a data acquisition unit 20.

The illustrated data acquisition unit 20 can serve as a central interface for receiving, preprocessing, and forwarding physiological data obtained from the sensors 14. In particular, the data acquisition unit 20 can be configured to receive sensor data 16 corresponding to one or more types of physiological data, including movement-related data that can be used for subsequent determination of movement asymmetry, movement variability, or related movement characteristics, and to perform one or more preprocessing, conditioning, and data preparation operations prior to downstream data analysis. The data acquisition unit 20 can be configured to reliably collect the sensor data 16 in real time or near real time from the sensors 14, which may include one or more contact-based or non-contact sensing devices. Upon receipt, the data acquisition unit 20 can perform preprocessing operations including, for example, signal conditioning (e.g., amplification, filtering, normalization, and artifact removal), noise reduction, baseline correction, smoothing, resampling, synchronization across multiple sensor streams, data formatting, and handling of missing or incomplete data. In some embodiments, the data acquisition unit 20 can perform data cleaning operations, including detection and removal or correction of corrupted, inconsistent, or outlier data points, interpolation or imputation of missing values, and standardization or normalization of data formats to facilitate subsequent processing. Where applicable, the data acquisition unit 20 can further perform digitization operations, including converting analog signals into digital representations, and can format or structure the sensor data 16 into one or more standardized or intermediate data representations suitable for downstream data processing pipelines. In embodiments involving image or video-based sensors and associated sensor data 16, the preprocessing can include frame extraction, resolution adjustment, image normalization, and temporal alignment of frame sequences.

The data acquisition unit 20 can additionally be optionally configured to apply or add temporal and contextual metadata to the sensor data 16, including for example timestamping, sequencing, and association with patient identifiers, session identifiers, or environmental context. The data acquisition unit 20 can further perform signal integrity verification operations, including error detection, consistency checks, calibration validation, and sensor status monitoring, to ensure reliability and quality of the acquired data. In one embodiment, the data acquisition unit 20 includes one or more processors or controllers configured to execute instructions for performing the foregoing operations, as well as memory or storage components for storing application software and sensor data, including both raw and preprocessed data. The data acquisition unit 20 can further include one or more communication modules configured to receive data from the sensors 14 and to transmit preprocessed sensor data 22 to downstream system components. Accordingly, the data acquisition unit 20 can generate preprocessed sensor data 22 that has been conditioned, cleaned, formatted, and prepared for subsequent analysis, thereby improving the accuracy, consistency, and reliability of downstream assessment operations performed by the system.

The preprocessed sensor data 22 can be provided to a pose estimation unit 30 configured to estimate or determine a pose of the infant, where pose refers to the spatial arrangement and orientation of one or more anatomical landmarks or body segments in two-dimensional or three-dimensional space at a given time instance. The pose estimation unit 30 can be configured to determine spatial representations of one or more portions or features of the infant’s body by detecting, localizing, and tracking selected anatomical landmarks over time. As used herein, the term “spatial representation” refers to a structured representation of the position, orientation, or arrangement of one or more objects or anatomical features within a physical space, including, without limitation, two-dimensional or three-dimensional coordinates, key point or landmark representations, skeletal models, or other data structures that describe spatial relationships or configurations over time. The anatomical landmarks can include identifiable external regions or reference points associated with anatomical features of the infant’s body that provide meaningful data for movement, posture, or physiological analysis. Such anatomical landmarks can include, for example, the head, shoulders, elbows, wrists, hips, knees, and ankles, as well as other regions such as the thoracic region (e.g., sternum and rib cage), abdominal region (e.g., umbilicus), cranial region (e.g., forehead), posterior torso (e.g., back), and peripheral extremities (e.g., upper arm, wrist, palm, thigh, ankle, foot, heel, or toe). In certain embodiments, the anatomical landmarks can correspond to identifiable reference points associated with joints, limbs, or body segments, and can be used to represent spatial relationships among such features.

The pose estimation unit 30 can be configured to process the preprocessed sensor data 22, which can include image data, video data, or other sensor-derived data, to generate pose data 32 representing spatial representations of the detected anatomical landmarks or body segments, including, for example, spatial positions, orientations, and, in some embodiments, temporal trajectories over time. The pose estimation unit 30 can employ one or more computational techniques, including computer vision algorithms, geometric modeling, and/or machine learning models, to infer the pose of the infant from the sensor data. The pose estimation unit 30 can be further configured to determine or infer a pose state of the infant based on the spatial representation of the detected anatomical landmarks or body segments. In particular, the pose estimation unit 30 can analyze the spatial arrangement and relative positions and orientations of the anatomical landmarks, as represented in the pose data 32, to optionally classify the infant’s pose state. The pose states can include, for example, supine, prone, side-lying, curled, or extended positions, which can be identified based on relative orientations and spatial relationships among the head, torso, and limbs. The pose data 32 can be output in a structured representation suitable for downstream processing by other units of the system 10, including movement extraction, symmetry assessment, or variance analysis. In some embodiments, the pose estimation unit 30 can generate the pose data 32 without performing temporal aggregation or higher-level statistical analysis of movement patterns.

The pose data 32 generated by the pose estimation unit 30 can be transmitted to a data cleaning and normalization unit 40 configured to refine, standardize, and validate the pose data 32 to generate normalized pose data 42 suitable for downstream movement analysis and assessment. The pose data 32 can represent spatial representations of detected anatomical landmarks or body segments, including positions, orientations, and, in some embodiments, temporal trajectories over time. The pose data 32 can be further processed by the pose estimation unit 30 or other units to determine or infer a pose state of the infant based on the spatial relationships among the anatomical landmarks. The data cleaning and normalization unit 40 can operate on such pose-domain representations to improve accuracy, consistency, and usability.

The data cleaning and normalization unit 40 can be configured to perform one or more data cleaning and normalization operations on the pose data 32 to generate normalized pose data 42 suitable for downstream processing. The data cleaning and normalization operations can include operations sufficient for improving quality, consistency, completeness, and comparability of data derived from spatial representations of anatomical landmarks. The data cleaning and normalization operations can include, for example, one or more data cleaning operations, such as detection and removal or correction of invalid, corrupted, or outlier pose points, handling of missing or occluded anatomical landmark data, interpolation or imputation of missing landmark positions, and smoothing of pose trajectories or time-series pose data to reduce noise and temporal instability. The data cleaning and normalization unit 40 can also apply filtering techniques to mitigate artifacts resulting from sensor inaccuracies or environmental conditions and can optionally enforce anatomical or kinematic constraints to improve consistency of the spatial representations of the anatomical landmarks.

The data cleaning and normalization operations can further include normalization operations to transform the pose data 32 into a standardized representation. The normalization operations can transform pose coordinates into a common reference frame, aligning poses relative to a reference anatomical feature or spatial origin (e.g., torso or center of mass), normalizing for scale, orientation, or camera perspective, and synchronizing the pose data across time or across multiple sensor data streams. The data cleaning and normalization unit 40 can also standardize data formats, coordinate systems, and data structures to ensure compatibility with downstream processing units. The data cleaning and normalization unit 40 can further perform validation and quality assessment operations, including consistency checks, confidence scoring, and verification of temporal continuity of the pose data 32. Based on the validation, the data cleaning and normalization unit 40 can flag or discard unreliable data segments or annotate the pose data with quality indicators. The data cleaning and normalization unit 40 can also generate temporally aligned and continuous spatial representations of the anatomical landmarks, thereby facilitating accurate detection and characterization of movement events by downstream units, including the movement extraction unit 50. In this manner, the normalized pose data 42 can represent a cleaned, standardized, temporally aligned, and quality-controlled representation of the spatial relationships among anatomical landmarks, suitable for subsequent processing. In certain embodiments, preprocessing of the raw sensor data 16 can be performed by the data acquisition unit 20, while the data cleaning and normalization unit 40 operates on pose-derived representations of the pose data 32.

The normalized pose data 42 can be received and processed by a movement extraction unit 50 configured to identify, segment, and characterize movement activity of the infant based on temporal analysis of the normalized pose data 42. The normalized pose data 42 can represent spatial representations of anatomical landmarks or body segments over time, and the movement extraction unit 50 can analyze the temporal spatial representations to detect and delineate movement activity associated with one or more anatomical regions, including individual joints, limbs, or combined body segments on the same or opposite sides of the body.

In certain embodiments, the movement extraction unit 50 can identify movement activity according to a hierarchical framework including discrete movement events, continuous movement states, and temporal movement patterns. The discrete movement events can include temporally bounded movements having identifiable onset and offset, such as limb displacements, trunk rotations, or head movements. The continuous movement states can include sustained conditions such as periods of reduced or absent motion or maintained postures. The temporal movement patterns can include sequences or repetitions of movements over time, such as repetitive behaviors, coordinated motion sequences, or restlessness.

The movement extraction unit 50 can further quantify the detected movement activity by generating movement feature data 52, which can include structured representations of movement activity derived from the normalized pose data 42. The movement feature data 52 can represent trajectories of anatomical landmarks or body segments and associated movement features. The movement features can include, for example, displacement, velocity, acceleration, angular motion, range of motion, duration, frequency, temporal spacing, and other kinematic or temporal characteristics.

In some embodiments, the movement extraction unit 50 can track trajectories of joints or body segments over time and extract time-series representations of movement activity for subsequent analysis. The movement extraction unit 50 can determine one or more movement features or descriptors for the identified movement activity, including per-region or per-joint feature vectors and temporal representations suitable for downstream processing.

The movement extraction unit 50 can also generate aggregated movement metrics based on the extracted features, including summary statistics or composite indicators representing characteristics of movement activity over selected time intervals. The movement extraction unit 50 can optionally perform preliminary categorization of movement types (e.g., gross versus fine motor activity, voluntary versus involuntary motion) and can identify recurring or structured movement activity without performing higher-level asymmetry comparison or variance quantification, which can be performed by downstream units.

The movement extraction unit 50 can optionally employ one or more computational techniques to perform the foregoing operations, including for example kinematic analysis of pose trajectories, time-series analysis, and signal processing techniques such as filtering, peak detection, thresholding, and segmentation algorithms for identifying movement onset and offset. In certain embodiments, machine learning models can be employed, including supervised or unsupervised models configured to detect, classify, or cluster movement activity. The models can include sequence models, temporal convolutional networks, recurrent neural networks, transformer-based temporal models, or other pattern recognition techniques. In some embodiments, rule-based or heuristic methods can be used to detect predefined movement activity based on thresholds or kinematic criteria. In this manner, the movement feature data 52 can represent structured, temporally derived characterizations of movement activity based on spatial relationships and trajectories of anatomical landmarks, thereby providing a basis for downstream analysis, including determination of temporal movement variability, inter-limb differences, and asymmetry measures.

In certain embodiments, the movement feature data 52 generated by the movement extraction unit 50 can provide a structured representation of movement activity sufficient for operation of the system 10, including detection, segmentation, and quantification of movement activity. In such embodiments, the system 10 can operate based on the movement feature data 52 to monitor movement and generate outputs, including visualizations or alerts, without requiring additional higher-level analytical units. The movement feature data 52 can be conveyed to one or more downstream units, including directly to the display unit 90, for presentation, visualization, and/or further analysis.

In other embodiments, one or more additional units, including a limb movement comparator unit 60, a cross-midline movement comparator unit 70, and a movement variance quantification unit 80, can be employed to provide enhanced analysis of the movement feature data 52. These units can perform comparative, relational, or aggregated analyses of movement activity, including intra-limb comparisons, inter-limb symmetry analysis, and variability quantification, thereby providing higher-level interpretation of movement characteristics.

The movement feature data 52, which represents structured representations of movement activity derived from spatial representations of anatomical landmarks, can be optionally conveyed to and processed by a limb movement comparator unit 60 and/or a cross-midline movement comparator unit 70. The limb movement comparator unit 60 can be configured to provide enhanced analysis of movement activity by evaluating movement activity of a selected limb of the infant through intra-limb analysis, including comparison of current or recent movement activity relative to prior movement activity of the same limb over time. The limb movement comparator unit 60 can operate on components of the movement feature data 52 associated with a given anatomical region or limb. The components can include trajectory data corresponding to movements of the anatomical landmarks or body segments over time and associated movement features that characterize movement activity, such as displacement, velocity, acceleration, range of motion, frequency, duration, and other kinematic or temporal descriptors. In certain embodiments, the limb movement comparator unit 60 can segment the movement feature data 52 by anatomical region and define one or more temporal comparison windows (e.g., sequential, sliding, or overlapping intervals) within a monitoring session.

The limb movement comparator unit 60 can perform temporal comparisons of movement activity across the defined intervals to detect changes, trends, anomalies, or deviations in movement activity of the selected limb over time. The comparisons can include determining statistical descriptors of movement activity, such as mean, variance, amplitude, and frequency, and evaluating differences in these descriptors between time windows or over time. The limb movement comparator unit 60 can further optionally apply time-series analysis and signal processing techniques, including filtering, spectral analysis, and feature differencing, to characterize temporal evolution of movement activity for the selected limb.

In certain embodiments, the limb movement comparator unit 60 can also assess variability and complexity of movement activity using entropy-based or related measures, including, for example, sample entropy, approximate entropy, permutation entropy, or fractal-based metrics. The entropy-based measures generally refer to quantitative techniques for evaluating the degree of irregularity, unpredictability, or complexity within a time-series representation of movement activity, such as variations in trajectories of anatomical landmarks over time. The measures can provide quantitative representations of movement regularity, predictability, or variability over time.

The limb movement comparator unit 60 can generate limb movement comparison data 62, which can include structured representations of temporal changes in movement activity for a selected limb, including comparative metrics, trend indicators, variability measures, and derived descriptors characterizing changes in movement activity over time. The limb movement comparison data 62 can be conveyed to one or more downstream units, including the display unit 90 and/or the movement variance quantification unit 80, for visualization, further comparative analysis, variance quantification, or assessment. In certain embodiments, the limb movement comparison data 62 can be processed independently to generate visualizations, alerts, or quantitative measures associated with movement activity of the selected limb. In other embodiments, the limb movement comparison data 62 can be combined with outputs from the cross-midline movement comparator unit 70 to enable integrated analysis of intra-limb and inter-limb movement activity, including symmetry and coordination assessment. In this manner, the limb movement comparator unit 60 can provide enhanced, time-dependent analysis of movement activity for individual limbs, supplementing the movement feature data 52 with comparative and trend-based insights while remaining optional within the overall system architecture.

Further, the illustrated detection and assessment system 10 can employ the optional cross-midline movement comparator unit 70 configured to receive and process the movement feature data 52, which represents structured representations of movement activity derived from spatial representations of anatomical landmarks. The cross-midline movement comparator unit 70 can be configured to evaluate movement activity across contralateral anatomical regions of the infant by comparing movement activity associated with opposing limbs or body segments (e.g., left arm relative to right arm, or left leg relative to right leg). According to one embodiment, the cross-midline movement comparator unit 70 can operate on components of the movement feature data 52 corresponding to contralateral regions. As used herein, the term “contralateral regions” refers to corresponding anatomical regions located on opposite sides of the body relative to a midline, such as left and right limbs or body segments. The components can include trajectory data corresponding to movements of anatomical landmarks or body segments over time and associated movement features that characterize movement activity, such as displacement, velocity, acceleration, range of motion, frequency, duration, and other kinematic or temporal descriptors. The cross-midline movement comparator unit 70 can align and compare such movement activity across the contralateral regions within defined temporal windows (e.g., synchronized, sliding, or overlapping intervals) to enable both instantaneous and time-based comparative analysis.

The cross-midline movement comparator unit 70 can also be configured to quantify inter-limb relationships by determining one or more comparative metrics, including differences, ratios, or normalized measures between corresponding movement features of the contralateral limbs. Additionally, the cross-midline movement comparator unit 70 can evaluate temporal coordination characteristics, including relative timing, phase differences, synchronization, and coupling between movements of opposing limbs. In certain embodiments, signal processing and time-series analysis techniques, including cross-correlation, phase analysis, and spectral comparison, can be employed to assess the degree of coordination or divergence between limbs.

The cross-midline movement comparator unit 70 can generate inter-limb comparison data 72, which can include structured representations of comparative metrics of movement activity between contralateral anatomical regions. The comparative metrics can include symmetry or asymmetry measures, coordination metrics, timing relationships, and relative differences in movement activity derived from the movement feature data 52. The inter-limb comparison data 72 can further include derived indicators representing degrees of bilateral similarity or divergence in movement activity over time. The inter-limb comparison data 72 can be conveyed to one or more downstream units, including for example directly to the display unit 90 and/or to the movement variance quantification unit 80, for visualization, further analysis, or evaluation. As such, the inter-limb comparison data 72 can be processed independently to generate visualizations, alerts, or quantitative measures associated with symmetry, coordination, or variability of movement activity. In other embodiments, the inter-limb comparison data 72 can be combined with the limb movement comparison data 62 generated by the limb movement comparator unit 60 for processing by the movement variance quantification unit 80, thereby enabling integrated analysis of intra-limb and inter-limb movement activity. In certain implementations, thresholds, rules, or criteria can be applied to the inter-limb comparison data 72, alone or in combination with the limb movement comparison data 62, to identify significant deviations in movement activity, which can be flagged for review or further processing by subsequent system components.

The detection and assessment system 10 can further include a movement variance quantification unit 80 configured to receive and process one or more inputs including limb movement comparison data 62 generated by the limb movement comparator unit 60 and inter-limb comparison data 72 generated by the cross-midline movement comparator unit 70. The movement variance quantification unit 80 can be configured to quantify variability, asymmetry, and distribution of movement activity across one or more anatomical regions of the infant based on such inputs, which are derived from spatial representations of anatomical landmarks.

In certain embodiments, the movement variance quantification unit 80 can process the limb movement comparison data 62 independently to quantify intra-limb variability of movement activity over time. This processing can include evaluation of temporal changes, trends, and variability of movement activity for individual limbs or anatomical regions. The movement variance quantification unit 80 can determine statistical and signal-based descriptors from the limb movement comparison data 62, including, for example, variance, standard deviation, coefficient of variation, entropy-based measures, and temporal fluctuation metrics that characterize variability of movement activity within a single limb. In this manner, the movement variance quantification unit 80 can generate localized variability representations indicative of stability, irregularity, or deviation of movement activity for individual limbs.

In certain embodiments, the movement variance quantification unit 80 can process the inter-limb comparison data 72 independently to quantify inter-limb asymmetry and coordination of movement activity across contralateral anatomical regions. This processing can include evaluation of symmetry or asymmetry between opposing limbs, phase relationships, timing differences, and relative differences in movement activity. The movement variance quantification unit 80 can thus determine asymmetry measures and coordination metrics, including normalized asymmetry indices, inter-limb difference metrics, and synchronization measures that characterize the degree of bilateral similarity or divergence in movement activity.

In other embodiments, the movement variance quantification unit 80 can process the limb movement comparison data 62 and the inter-limb comparison data 72 in combination to generate integrated representations of movement variability and asymmetry across the body. In such embodiments, the movement variance quantification unit 80 can aggregate intra-limb variability information with inter-limb asymmetry information to characterize both localized and distributed movement activity patterns. This combined processing can enable identification of relationships between intra-limb variability and inter-limb asymmetry, including cases where localized irregularity in one limb contributes to or correlates with bilateral asymmetry.

The movement variance quantification unit 80 can further integrate temporal information across defined time windows to evaluate changes in movement activity over time, including trends, stability, progression, and fluctuation. In certain embodiments, the movement variance quantification unit 80 can generate whole-body movement variability representations by aggregating limb-level and inter-limb data to characterize spatial and temporal disparities across multiple body segments, including grouped anatomical regions such as upper extremities relative to lower extremities. In some embodiments, the movement variance quantification unit 80 can also incorporate spatial orientation information derived from the pose data 32, including positional coordinates and rotational characteristics of body segments, to evaluate orientation-dependent variability of movement activity across one or more planes of motion (e.g., sagittal, frontal, and transverse). The rotational characteristics can include, for example, pitch, roll, and yaw, or other angular representations.

The movement variance quantification unit 80 can generate movement variability data 82 representing quantified characteristics of movement activity based on one or more of the limb movement comparison data 62 and the inter-limb comparison data 72. In particular, the movement variability data 82 can represent at least one of variability, asymmetry, or coordination of the movement activity. When generated from the limb movement comparison data 62, the movement variability data 82 can include metrics characterizing variability of movement activity for one or more anatomical regions over time. When generated from the inter-limb comparison data 72, the movement variability data 82 can include metrics characterizing asymmetry or coordination of movement activity across contralateral anatomical regions. When generated from combined processing of the limb movement comparison data 62 and the inter-limb comparison data 72, the movement variability data 82 can include integrated metrics characterizing variability, asymmetry, or coordination of movement activity across multiple anatomical regions, including relationships between intra-limb variability and inter-limb asymmetry. In certain embodiments, the movement variability data 82 can further include aggregated or composite metrics representing overall movement activity characteristics of the infant over a selected time interval. The movement variability data 82 can be suitable for downstream processing, visualization, alert generation, or clinical assessment.

Further, the detection and assessment system 10 can include a display unit 90 configured to receive and present outputs generated by one or more upstream system components in a human-readable format. The display unit 90 can be configured to receive data from one or more of the movement extraction unit 50, the limb movement comparator unit 60, the cross-midline movement comparator unit 70, and the movement variance quantification unit 80, and to present corresponding representations of movement activity, comparative analyses, and variability or asymmetry metrics to a user.

In certain embodiments, the display unit 90 can directly receive and present movement feature data 52 generated by the movement extraction unit 50, which represents structured representations of movement activity derived from spatial representations of the anatomical landmarks. The display unit 90 can display the movement activity through graphical representations including trajectories of anatomical landmarks or body segments, time-series representations of movement activity, and quantitative displays of movement features such as displacement, velocity, acceleration, frequency, and duration. The display unit 90 can also present indicators of detected movement activity, including movement events, states, or patterns, and can generate alerts 92 based on predefined thresholds or conditions associated with the movement feature data 52.

In certain embodiments, the display unit 90 can directly receive and present limb movement comparison data 62 generated by the limb movement comparator unit 60. The display unit 90 can present intra-limb comparative analyses of movement activity, including temporal trends, variability measures, anomaly indicators, and changes in movement activity over time for individual limbs or anatomical regions. The display unit 90 can further generate alerts 92 based on deviations, irregularities, or threshold conditions identified in the limb movement comparison data 62. In certain embodiments, the display unit 90 can directly receive and present inter-limb comparison data 72 generated by the cross-midline movement comparator unit 70. The display unit 90 can present inter-limb comparative analyses of movement activity, including symmetry or asymmetry measures, coordination metrics, timing relationships, and synchronization indicators between contralateral anatomical regions. The display unit 90 can further generate alerts 92 based on detected asymmetry, coordination irregularities, or predefined criteria associated with inter-limb movement activity.

In certain embodiments, the display unit 90 can receive and present movement variability data 82 generated by the movement variance quantification unit 80. The movement variability data 82 can be generated based on processing of the limb movement comparison data 62, the inter-limb comparison data 72, or a combination of both. When generated from the limb movement comparison data 62, the movement variability data 82 can include intra-limb variability metrics and temporal variability indicators. When generated from the inter-limb comparison data 72, the movement variability data 82 can include asymmetry measures, coordination metrics, and inter-limb comparison indicators. When generated from combined processing of the output data 62 and 72, the movement variability data 82 can include integrated representations of movement activity, including composite variability indices, asymmetry scores, whole-body variability descriptors, and derived indicators reflecting relationships between intra-limb variability and inter-limb asymmetry.

The display unit 90 can be configured to generate one or more graphical user interfaces that visually represent such information, including, without limitation, charts, graphs, heatmaps, trajectory plots, skeletal overlays, time-series plots, or dashboard-style interfaces that depict movement activity, variability, asymmetry indices, and spatial or positional evolution of body segments over time. The display unit 90 can present both instantaneous and longitudinal views of movement activity, including real-time monitoring displays and historical trend visualizations over selected time intervals. In certain embodiments, the display unit 90 can present derived indices or composite scores in summarized or aggregated formats.

In some embodiments, the display unit 90 can present alerts 92 generated by one or more upstream units or generated by the system 10 based on analysis of the data 52, 62, 72, and/or 82 when predefined conditions or thresholds are met, including conditions associated with movement activity, variability, asymmetry, or changes in movement activity over time. As used herein, the term “alert,” in either singular or plural form, can refer to any notification, indication, signal, message, flag, or communication generated by the system in response to one or more detected conditions, criteria, or events. The alert can be generated when one or more predefined thresholds, rules, patterns, or model-based determinations are satisfied, including conditions associated with movement variability, asymmetry, or deviations in movement activity. The alert can be presented in one or more forms, including visual, auditory, tactile, or electronic formats, such as graphical indicators (e.g., icons, color changes, overlays, or highlighted regions), textual messages, numerical indicators, alarms, tones, vibrations, or other user-perceivable signals.

In addition to on-screen presentation, the alerts 92 can be transmitted or communicated to one or more systems, devices, or recipients, including a clinical care provider or critical care personnel, via one or more communication channels, including mobile devices, wearable devices, monitoring systems, electronic health record (EHR) systems, or remote computing platforms. In some embodiments, the alerts 92 can include varying levels of severity, priority, or classification and can be associated with contextual information, including underlying data, time of occurrence, and associated movement activity metrics or indices. The alerts can be generated in real time or based on analysis of historical or aggregated data. In this manner, the display unit 90 can facilitate interpretation of movement activity and support monitoring and assessment workflows by presenting structured and derived information from multiple system components in an accessible format without performing underlying movement computation or analysis operations.

In operation, the detection and assessment system 10 can be configured to receive sensor data 16 associated with an infant from one or more sensors 14. The sensor data 16 can include, for example, image data, video data, depth data, or other sensor-derived data representative of the infant’s position, posture, or movement. The sensor data 16 can be received and preprocessed by the data acquisition unit 20 to generate preprocessed sensor data 22, which can include conditioned, filtered, and temporally synchronized data suitable for downstream processing.

The preprocessed sensor data 22 can be provided to the pose estimation unit 30, which can be configured to determine spatial representations of anatomical landmarks or body segments of the infant. The pose estimation unit 30 can generate pose data 32 representing positions, orientations, and, in some embodiments, temporal trajectories of the anatomical landmarks over time. The pose data 32 can be provided to the data cleaning and normalization unit 40, which can generate normalized pose data 42 by performing one or more operations including noise filtering, interpolation of missing data, smoothing, and normalization to a common reference frame or coordinate system. The normalized pose data 42 can be provided to the movement extraction unit 50, which can be configured to identify, segment, and characterize movement activity of the infant based on temporal analysis of spatial representations of the anatomical landmarks. The movement extraction unit 50 can generate movement feature data 52, which can include structured representations of movement activity, including trajectories and associated movement features such as displacement, velocity, acceleration, frequency, duration, and other kinematic or temporal descriptors. The movement feature data 52 can represent movement activity in terms of discrete movement events, continuous movement states, and temporal movement patterns.

The movement feature data 52 can be conveyed to one or more downstream units for further analysis. The limb movement comparator unit 60 can receive the movement feature data 52 and generate limb movement comparison data 62 by performing intra-limb analysis of movement activity, including temporal comparison of movement activity for individual limbs or anatomical regions. The cross-midline movement comparator unit 70 can receive the movement feature data 52 and generate inter-limb comparison data 72 by performing inter-limb analysis of movement activity across contralateral anatomical regions, including evaluation of symmetry, coordination, and timing relationships.

In selected embodiments, the limb movement comparison data 62 and the inter-limb comparison data 72 can be processed independently or in combination. The movement variance quantification unit 80 can receive one or both of the limb movement comparison data 62 and the inter-limb comparison data 72 and can generate movement variability data 82 representing variability, asymmetry, and distribution of movement activity across one or more anatomical regions. When processing the limb movement comparison data 62 independently, the movement variance quantification unit 80 can quantify intra-limb variability of movement activity. When processing the inter-limb comparison data 72 independently, the movement variance quantification unit 80 can quantify inter-limb asymmetry and coordination. When processing the output data 62 and 72 in combination, the movement variance quantification unit 80 can generate integrated representations of movement variability and asymmetry, including composite indices and whole-body movement descriptors.

The outputs of one or more of the movement extraction unit 50, the limb movement comparator unit 60, the cross-midline movement comparator unit 70, and the movement variance quantification unit 80 can be conveyed to the display unit 90. The display unit 90 can present the movement feature data 52, limb movement comparison data 62, inter-limb comparison data 72, and/or movement variability data 82 in one or more user interfaces. The display unit 90 can generate visual representations of movement activity, comparative analyses, and variability or asymmetry metrics, including trajectory plots, time-series representations, heatmaps, and dashboard-style visualizations. The display unit 90 can also generate alerts 92 based on one or more of the output data 52, 62, 72, and/or 82 when predefined conditions or thresholds are satisfied, including conditions associated with movement variability, asymmetry, or deviations in movement activity over time. The alerts 92 can be presented visually or communicated to one or more external systems or users to facilitate monitoring and assessment of the infant.

In this manner, the detection and assessment system 10 can progressively transform sensor-derived data into structured, machine-interpretable representations of movement activity and associated variability and asymmetry metrics. More specifically, the system 10 can transform the raw sensor data 16 into preprocessed sensor data 22, which can be used to generate pose data 32 representing spatial representations of anatomical landmarks. The pose data 32 can be further processed to generate normalized pose data 42, which can be analyzed to produce movement feature data 52 representing structured representations of movement activity. The movement feature data 52 can be further processed to generate limb movement comparison data 62 and inter-limb comparison data 72 representing intra-limb and inter-limb analyses of movement activity. The movement variance quantification unit 80 can receive and process the data 62 and/or 72, independently or in combination, to generate movement variability data 82 representing variability and asymmetry of movement activity across one or more anatomical regions over time. The movement variability data 82 can include, for example, quantified asymmetry measures, variability metrics, temporal trend indicators, and composite indices representing differences in movement activity across anatomical regions and time intervals. Accordingly, the system 10 converts unstructured or semi-structured sensor-derived physiological data into structured representations of movement activity and derived variability and asymmetry metrics, thereby enabling objective, quantitative characterization of movement behavior that is not directly observable from the raw sensor data alone. In this manner, the system 10 supports real-time and longitudinal assessment of infant movement and facilitates monitoring, evaluation, and clinical decision-making workflows.

The movement feature data 52, limb movement comparison data 62, inter-limb comparison data 72, and movement variability data 82 can be provided to the display unit 90, which functions as a presentation layer to convey the processed information in a human-readable format. The display unit 90 can generate graphical representations such as charts, trajectory plots, skeletal overlays, heatmaps, and time-series visualizations to depict movement characteristics, comparative metrics, and variability measures over time. In some embodiments, the display unit 90 can also present alerts or notifications generated by upstream units when predefined thresholds or conditions are satisfied. In this manner, the system 10 provides a structured transformation of raw sensor data into progressively refined representations of posture, movement, comparative relationships, and variability metrics, thereby facilitating monitoring, interpretation, and assessment of infant movement in a clinical or care setting.

The sensors 14 employed by the detection and assessment system 10 can be arranged in a sensor subsystem that is configured to acquire the physiological data associated with movement, posture, and positional characteristics of the infant 12. The sensor subsystem can utilize various types of sensors, including, without limitation, accelerometers, gyroscopes, inertial measurement units (IMUs), force or pressure sensors, and optical sensing systems, such as camera-based or depth-based tracking systems. The selection and configuration of sensors can be based on desired sensitivity, accuracy, temporal resolution, and suitability for use in clinical environments, including neonatal intensive care unit (NICU) and pediatric intensive care unit (PICU) settings. In certain embodiments, sensor placement can be selected or optimized to capture movement of specific anatomical regions or body segments, and can be configured to enable comprehensive detection of limb movement, body position, and whole-body motion.

Embodiments of the detection and assessment system 10 of the present invention provide improvements to the field of neonatal and infant monitoring and assessment technology by enabling objective, non-invasive, and continuous quantification of movement characteristics that are not directly observable from raw sensor data. In particular, the system 10 processes physiological data acquired from one or more sensors 14 and transforms the sensor data 16 into structured representations of posture, movement, comparative relationships, and quantified variability and asymmetry measures. Through the coordinated operation of the data acquisition unit 20, pose estimation unit 30, data cleaning and normalization unit 40, movement extraction unit 50, comparator units 60 and 70, and movement variance quantification unit 80, the detection and assessment system 10 generates movement variability data 82 that provides clinically meaningful, quantitative indicators of movement behavior of the infant across anatomical regions and over time.

This data transformation enables a practical application in a healthcare environment by improving the ability to monitor, assess, and track infant movement in real time or near real time. For example, by generating quantitative asymmetry measures and variability metrics across contralateral limbs, limb groups, and whole-body movement, the detection and assessment system 10 facilitates earlier and more consistent identification of deviations in movement characteristics, supports longitudinal tracking of motor development, and enhances the repeatability of movement assessment compared to subjective observation. The detection and assessment system 10 further enables continuous monitoring and automated analysis within clinical settings, such as NICUs and PICUs, thereby improving workflow efficiency and expanding accessibility of movement assessment without requiring specialized expertise to interpret raw sensor signals.

In addition to improving a field of technology, embodiments of the present invention also improve the functioning of computing systems used to process sensor-derived physiological data. Conventional computing systems processing raw sensor or image data are limited in their ability to directly extract clinically relevant movement information without substantial manual interpretation or ad hoc processing. In contrast, the detection and assessment system 10 implements a structured, multi-stage data processing pipeline that progressively converts unstructured or semi-structured sensor data 16 into increasingly refined and machine-interpretable representations, including preprocessed sensor data 22, pose data 32, normalized pose data 42, movement feature data 52, comparison data 62 and 72, and movement variability data 82.

This structured data transformation improves computer functionality in several ways. First, the system 10 employs specialized data representations, such as anatomical landmark-based pose data and normalized coordinate frameworks, that reduce data dimensionality and enable efficient downstream computation. Second, the system 10 improves the accuracy and robustness of computational analysis through data cleaning, normalization, and synchronization operations that mitigate noise, missing data, and sensor inconsistencies. Third, the system 10 enables efficient temporal and spatial analysis by organizing data into time-series structures and comparison windows, allowing for scalable computation of movement features, comparative metrics, and variability measures. Fourth, the system 10 produces compact, structured outputs, such as asymmetry indices, variability metrics, and composite scores, that reduce computational overhead for subsequent processing, storage, and visualization, while preserving clinically relevant information.

Accordingly, the detection and assessment system 10 of the present invention is not directed to the mere collection or analysis of data, but instead to a specific, technologically grounded process that transforms sensor-derived physiological data into new and useful representations that improve both the operation of computing systems and the capabilities of neonatal monitoring technology. These improvements are achieved through a particular arrangement of system components and data processing operations that yield concrete, practical benefits in real-world clinical environments.

According to another embodiment, the detection and assessment system 10 can further include or be operatively coupled to an artificial intelligence assistant (AI assistant) configured to receive and process one or more outputs generated by the system 10 and to generate corresponding responses, analyses, or recommendations. The AI assistant can operate as an auxiliary or downstream component that enhances interpretation, presentation, and utilization of data generated by the system without replacing or altering the core data processing pipeline described herein.

In one embodiment, the AI assistant can receive one or more of the movement feature data 52, limb movement comparison data 62, inter-limb comparison data 72, movement variability data 82, and/or alerts generated by the system 10. Based on these inputs, the AI assistant can generate outputs including, for example, natural language summaries, explanations of movement characteristics, descriptions of temporal trends, comparative analyses across time intervals or anatomical regions, or contextualized interpretations of quantified asymmetry measures and variability metrics.

The AI assistant can be configured to provide user-interactive functionality, including for example responding to user queries related to movement characteristics of the infant, generating on-demand reports, or presenting explanations of system outputs in a human-readable format. For example, the AI assistant can respond to queries regarding changes in movement variability over time, differences between contralateral limbs, or trends associated with previously generated movement variability data 82. The AI assistant can further generate structured or semi-structured outputs, such as summaries, tables, or formatted reports, based on the underlying data produced by the system 10.

In some embodiments, the AI assistant can operate in conjunction with the display unit 90 to present its outputs via one or more user interfaces, including graphical interfaces, dashboards, or conversational interfaces. Additionally or alternatively, the AI assistant can communicate its outputs to one or more external systems, including computing systems, mobile devices, or systems within a healthcare facility.

In certain embodiments, the AI assistant can employ one or more computational models, including machine learning models, generative language models, or other artificial intelligence techniques, to generate selected outputs. The AI assistant can optionally incorporate contextual information, including historical movement data, prior system outputs, or user-provided inputs, to generate more informative or context-aware responses. In other embodiments, the AI assistant can also be configured to generate recommendations or suggested actions based on detected conditions or patterns in the data, such as significant changes in movement variability or asymmetry measures. The recommendations can be provided for review by a clinical care provider or other user and can be used to support monitoring and assessment workflows.

In this manner, the AI assistant can enhance the usability, interpretability, and accessibility of the outputs generated by the detection and assessment system 10 by converting structured movement data and quantified metrics into user-consumable formats and interactive outputs. The AI assistant can thus serve as an interface layer between the system-generated data and end users, facilitating efficient interpretation and utilization of movement-related information without altering the underlying computation of movement features, comparison data, or movement variability data.

The present invention is also directed to another embodiment of an infant detection and assessment system 100 configured to provide non-invasive evaluation and assessment of infant (e.g., neonatal) health and development in a healthcare facility. In certain embodiments, the system 100 can employ one or more non-contact sensing modalities to capture sensor data associated with the infant while reducing stress and discomfort and enabling point-of-care assessment. The detection and assessment system 100 can be configured to process multimodal sensor data 16 to generate indicators, classifications, and/or scores associated with multiple assessment domains, including neuromotor characteristics, neuromuscular development, gaze-related behavior, posture-related characteristics (e.g., spinal alignment or curvature), anti-gravity movement, and physiological states. In certain embodiments, the detection and assessment system 100 can convert the sensor data 16 into movement-related data by identifying and characterizing motion of the infant based on spatial and temporal analysis of pose and sensor-derived inputs. The movement-related data can then be categorized into one or more movement classifications representing movement types, states, or patterns, and, in some embodiments, can be further processed to generate one or more scores or indices associated with the infant’s movement or physiological characteristics.

In certain embodiments, the detection and assessment system 100 can employ a pose estimation unit configured to determine spatial characteristics of the infant’s body, including posture and movement, using one or more computational models trained on infant-related data. The detection and assessment system 100 can further employ one or more machine learning models to process pose data and associated sensor-derived inputs to generate structured, quantitative or semi-quantitative outputs, including classification data, health-related scores, and associated confidence values. The outputs can be derived based on learned relationships from training data, including, in certain embodiments, labeled or reference datasets.

For enhanced flexibility, the detection and assessment system 100 can optionally integrate additional sensors, including thermal sensors, physiological sensors, or other sensing modalities, to augment the captured data and improve the robustness of the generated outputs. The detection and assessment system 100 can be configured for use in point-of-care environments in the healthcare facility to facilitate efficient assessment, monitoring, and evaluation of infant movement and physiological characteristics, including presentation of the classification and scoring outputs via one or more display interfaces. In this manner, the detection and assessment system 100 can transform the multimodal sensor data into structured, machine interpretable representations, including movement data, categorized outputs, and optional scores, that support monitoring and analysis of infant health and development based on the captured data.

The present invention is directed to another embodiment of a detection and assessment system 100 for assessing infant health and development at the point of care in infants, such as neonates, in a healthcare facility. The detection and assessment system 100 employs units that are similar to the units described herein in connection with the detection and assessment system 10 of FIG. 1, and like reference numerals indicate like parts throughout the various views. Further, in the detection and assessment system 100, the data acquisition unit 20, the pose estimation unit 30, the data cleaning and normalization unit 40, and the movement extraction unit 50 can operate in a manner consistent with the corresponding units described above, while optionally being configured to generate additional or alternative data representations or types for use in model-based assessment of infant health and development.

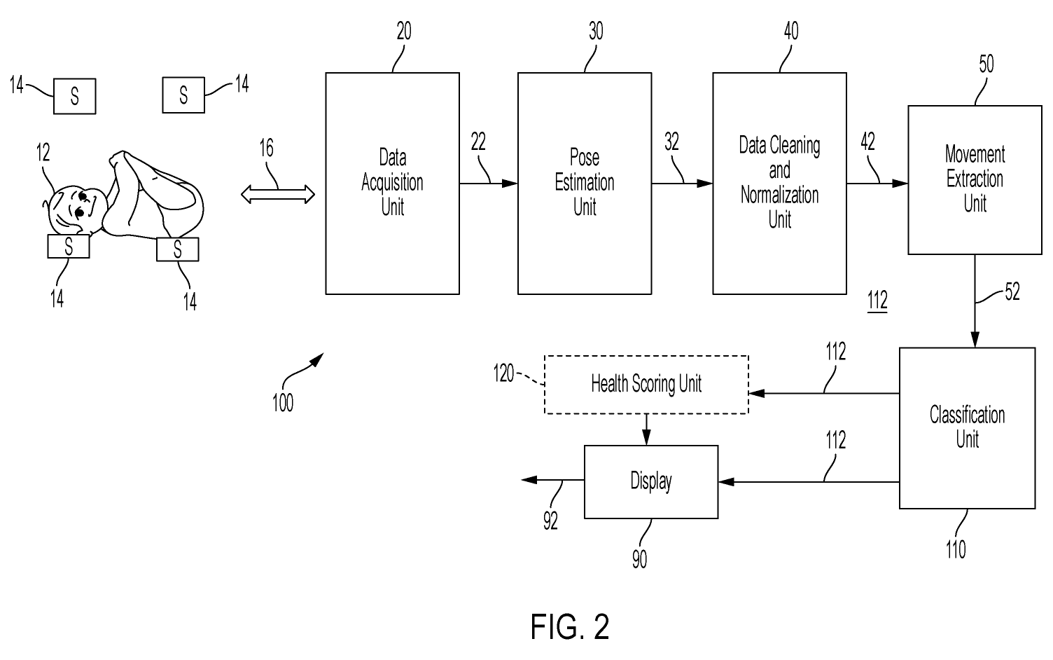

According to one embodiment, and as shown in FIG. 2, the detection and assessment system 100 of the present invention can employ one or more sensors 14 that can be positioned to capture and detect selected types of physiological data of the infant 12. Those of ordinary skill in the art will readily recognize that a single sensor or multiple sensors can be employed in the current system. For ease of description and for the sake of simplicity, the system is described as employing multiple sensors. The sensors 14 can be arranged at selected locations based on the type of sensors being employed. The sensors 14 can include, for example, contact (e.g., wearable) or non-contact sensors, such as for example accelerometers, pressure sensors, force sensors, thermal sensors including contact thermometers, wearable inertial measurement units, cameras including thermal cameras, or other suitable types of sensors. The sensors 14 can be strategically placed on or about the infant 12 to capture selected types of physiological data, such as body movement data, inertial data, and physiological state data, such as heart rate, blood oxygen levels, respiratory rate, and the like. The sensors 14 can also include cameras to capture image and video data of the infant, as well as thermal cameras to capture thermal data. The placement of the sensors 14 can be optimized to capture a comprehensive range of infant information, including body movement data, pose data, gaze related information, strength related data, and the like. The sensors 14 can generate sensor data 16 indicative of selected physiological data. The data can be processed to determine neuromotor function, neuromuscular development, gaze patterns, spinal curvature, anti-gravity strength, and/or physiological states.

With continued reference to FIG. 2, the illustrated data acquisition unit 20 can be configured to receive sensor data 16 from one or more sensors 14 and to generate preprocessed sensor data 22 suitable for downstream processing within the system 100. In certain embodiments, the data acquisition unit 20 can operate in a manner consistent with that described above in connection with FIG. 1, including performing preprocessing operations such as signal conditioning, filtering, synchronization, and formatting of sensor data. In some embodiments, the data acquisition unit 20 can perform data cleaning operations, including detection and removal or correction of corrupted, inconsistent, or outlier data points, interpolation or imputation of missing values, and standardization or normalization of data formats to facilitate subsequent processing. In the current embodiment, the data acquisition unit 20 can be further configured to handle and integrate multiple types of sensor data 16, including, for example, image data, video data, inertial measurement data, gaze-related data, thermal imaging data, and other physiological or environmental data associated with the infant. In some embodiments, the gaze-related data can include eye position, gaze direction, or visual attention indicators, and the thermal imaging data can include spatial temperature distributions or thermal gradients associated with the infant. The data acquisition unit 20 can optionally perform data fusion or temporal alignment of the data to generate unified data representations. The data acquisition unit 20 can generate preprocessed sensor data 22 that includes structured or semi-structured data representations derived from the sensor data 16. In addition to the representations described above, the preprocessed sensor data 22 can also include temporally aligned data sequences, multimodal data representations, and, in some embodiments, data formatted or configured for use as input to one or more computational models, including machine learning models.

With further reference to FIG. 2, the pose estimation unit 30 can be configured to receive the preprocessed sensor data 22 and to generate pose data 32 representing spatial characteristics of the infant’s body. In certain embodiments, the pose estimation unit 30 can operate in a manner consistent with that described above with respect to FIG. 1, including identifying and tracking anatomical landmarks corresponding to joints or body segments and generating spatial representations of the anatomical landmarks in two-dimensional or three-dimensional space. The pose estimation unit 30 can be configured to determine the spatial representations of the anatomical landmarks (e.g., portions or features of the infant’s body) by detecting, localizing, and tracking the landmarks over time. The anatomical landmarks can include identifiable external regions or reference points associated with anatomical features of the infant’s body that provide meaningful data for movement, posture, or physiological analysis.

The pose estimation unit 30 can optionally employ one or more computational models, including machine learning models, to estimate pose based on the preprocessed sensor data 22. The models can include, for example, computer vision models, keypoint detection models, transformer-based models, sequence models, or multimodal models configured to process image data, video data, depth data, gaze-related data, thermal data, or other sensor-derived inputs to detect and track anatomical landmarks of the infant over time. The pose estimation unit 30 can analyze the spatial arrangement and relative positions and orientations of the anatomical landmarks to spatially reconstruct the orientation of selected body segments and to optionally classify the infant’s pose state. The pose states can include, for example, supine, prone, side-lying, curled, or extended positions, which can be identified based on relative orientations and spatial relationships among the head, torso, and limbs. The pose estimation unit 30 can operate in a continuous or event-driven manner to detect transitions between pose states over time and to generate temporal pose trajectories representing movement of anatomical landmarks across successive time intervals.

The pose estimation unit 30 can generate pose data 32 that includes or represents, for example, the spatial representations of detected anatomical landmarks or body segments, landmark coordinates, joint positions, inter-joint spatial relationships, pose-state information, and temporal trajectory data over time. In some embodiments, the pose estimation unit 30 can further generate derived or encoded representations of pose information or state, including normalized pose vectors, feature embeddings, or other model-ready representations suitable for downstream processing, to determine or infer a pose state of the infant based on the spatial relationships among the anatomical landmarks. In certain embodiments, the pose estimation unit 30 can also optionally generate confidence values or probability measures associated with detected landmarks or inferred pose states, thereby providing an indication of reliability of the pose estimation. The pose data 32 can be transmitted to the data cleaning and normalization unit 40 and, in certain embodiments, to one or more downstream units or model-based assessment components of the system 100 for further analysis. In this manner, the pose estimation unit 30 can provide both geometric pose representations and machine learning-derived feature representations that support subsequent movement analysis and predictive assessment within the system 100.

The data cleaning and normalization unit 40 can be configured to perform one or more data cleaning and normalization operations on the pose data 32 and, in certain embodiments, one or more portions of the preprocessed sensor data 22, to generate normalized pose data 42 suitable for downstream processing. In certain embodiments, the data cleaning and normalization unit 40 can operate in a manner consistent with that described above with respect to FIG. 1, including refining, standardizing, and validating pose-related data to improve accuracy, consistency, and reliability.

In the current embodiment, the data cleaning and normalization unit 40 can be further configured to process combined data representations, including pose data 32 and selected portions of the preprocessed sensor data 22. The inclusion of the preprocessed sensor data 22 can enable the data cleaning and normalization unit 40 to align, synchronize, and normalize multiple data modalities, thereby facilitating integrated analysis across different types of sensor-derived information. For example, certain physiological or contextual information, such as gaze-related data, thermal data, inertial data, or image-derived features, may not be fully represented within the pose data 32 and can therefore be incorporated from the preprocessed sensor data 22 to provide a more comprehensive representation of the infant’s state.

The data cleaning and normalization unit 40 can perform one or data cleaning and normalization operations on the received data, including, without limitation, filtering or removal of invalid or noisy data points, interpolation or imputation of missing values, smoothing of temporal data, and transformation of data into a common coordinate system or reference frame. When multiple data sources are employed, the data cleaning and normalization unit 40 can further perform temporal alignment and synchronization across modalities to ensure consistency of data representations over time.

In certain embodiments, the data cleaning and normalization unit 40 can further generate standardized and model-ready data representations by performing operations such as feature scaling, normalization of feature values, encoding of categorical or state-based data, and segmentation of data into temporal windows. By optionally incorporating both pose data 32 and selected preprocessed sensor data 22, the data cleaning and normalization unit 40 can generate normalized pose data 42 that includes additional multimodal representations, normalized feature vectors, and temporally aligned data structures suitable for input into downstream units, including the movement extraction unit 50 and, in certain embodiments, one or more machine learning models. The normalized pose data 42 can thus be a temporally aligned and continuous spatial representations of the anatomical landmarks, thereby facilitating accurate detection and characterization of movement activity. In this manner, the normalized pose data 42 can represent a cleaned, standardized, temporally aligned, and quality-controlled representation of the spatial relationships among anatomical landmarks, suitable for subsequent processing. In this manner, the data cleaning and normalization unit 40 can provide an integrated data preparation layer that leverages both pose-based representations and additional optional sensor-derived information to generate consistent, normalized, and model-ready data representations for subsequent kinematic analysis and predictive modeling within the system 100.

The detection and assessment system 100 can also include the movement extraction unit 50 that can be configured to receive the normalized pose data 42 and to generate movement feature data 52 representing motion-related characteristics of the infant. The normalized pose data 42 can represent spatial representations of anatomical landmarks or body segments over time, and the movement extraction unit 50 can analyze the temporal spatial representations to detect and delineate movement activity associated with one or more anatomical regions, including individual joints, limbs, or combined body segments on the same or opposite sides of the body. In certain embodiments, the movement extraction unit 50 can operate in a manner consistent with that described above with respect to FIG. 1, including identifying and characterizing motion-related activity based on spatial and temporal analysis of pose data.

The movement extraction unit 50 can be configured to analyze the normalized pose data 42, and, in certain embodiments, combined data derived from the pose data 32 and selected portions of the preprocessed sensor data 22, to identify and characterize movement activity of the infant. The movement activity can include, for example, discrete movement events, continuous movement states, and temporal movement patterns derived from temporal changes in spatial representations of anatomical landmarks. The movement extraction unit 50 can detect discrete movement events corresponding to movement of individual or combined anatomical landmarks, joints, limbs, or body segments, including, fore xample, limb flailing, trunk rotation, head turning, postural shifts, repetitive movements, or periods of relative inactivity. In certain embodiments, the movement extraction unit 50 can identify continuous movement states, such as sustained postures or reduced motion conditions, and temporal movement patterns, including sequences or repetitions of movement activity over time.

In certain embodiments, the movement extraction unit 50 can perform temporal and/or spatial analysis of the spatial representations of the anatomical landmarks to track trajectories of the anatomical landmarks and to characterize the movement activity over time. The movement extraction unit 50 can also derive one or more movement features associated with the movement activity, including, for example, displacement, velocity, acceleration, angular motion, amplitude, duration, and frequency of movement. The movement extraction unit 50 can further generate temporal features, including timestamps, movement intervals, and frequencies associated with detected movement activity.

In some embodiments, the movement extraction unit 50 can generate structured feature representations derived from spatial representations of the anatomical landmarks that are suitable for downstream computational processing. The feature representations can include aggregated feature vectors, temporally segmented data windows, or encoded feature representations that capture characteristics of movement activity over defined time intervals. The feature representations can be derived from temporal changes in the spatial representations of the anatomical landmarks and can encode relationships between positions, orientations, and trajectories of the anatomical landmarks over time. When multiple data sources are employed, the movement extraction unit 50 can generate combined feature representations that incorporate movement features derived from the spatial representations of the anatomical landmarks together with additional sensor-derived features, such as gaze-related indicators, thermal patterns, or inertial signals. In this manner, the movement extraction unit 50 can integrate landmark-based spatial representations with complementary data modalities to enhance characterization of the movement activity.

The movement extraction unit 50 can generate movement feature data 52 having structured representations of movement activity, including representations of discrete movement events, continuous movement states, temporal movement patterns, trajectories of anatomical landmarks, movement features, and associated temporal characteristics. The movement feature data 52 can thus represent structured, machine-interpretable representations derived from spatial representations of anatomical landmarks and associated sensor-derived inputs. The movement extraction unit 50 thus provides a feature generation layer that transforms normalized pose data, representing spatial representations of anatomical landmarks, and associated sensor-derived inputs into structured representations of movement activity and model-ready feature sets suitable for downstream classification, comparative analysis, and predictive modeling.

The movement feature data 52 can be provided as input to one or more downstream units, including a classification unit 110 configured to classify movement activity of the infant based on the generated feature representations. In certain embodiments, the classification unit 110 can be configured to receive the movement feature data 52, and optionally one or more portions of the normalized pose data 42 and/or the preprocessed sensor data 22, and to generate classification data 112 representing categorized or inferred characteristics of the movement activity or physiological condition of the infant. The classification unit 110 can be configured to classify the movement activity into one or more categories, including categories corresponding to discrete movement events, continuous movement states, or temporal movement patterns derived from temporal changes in the spatial representations of the anatomical landmarks. In certain embodiments, the classification data 112 can further include inferred indicators associated with physiological or developmental conditions based on the classified movement activity. In this manner, the classification unit 110 can process structured representations of movement activity derived from spatial representations of anatomical landmarks, together with optionally integrated sensor-derived inputs, to generate higher-level categorizations or inferences suitable for downstream analysis, monitoring, or clinical assessment.

In certain embodiments, the classification unit 110 can employ one or more computational models, including machine learning models, to analyze the received data and to classify movement activity of the infant based on the movement feature data 52. As described herein, the movement activity can include discrete movement events, continuous movement states, or temporal movement patterns derived from temporal changes in spatial representations of anatomical landmarks. The models can include, for example, transformer-based models, recurrent neural networks (RNNs), temporal convolutional models, or other models configured to process time-series or multimodal data. In certain embodiments, the classification unit 110 can operate on temporally structured data, including sequences of movement feature data 52 derived from spatial representations of anatomical landmarks, to identify patterns, relationships, or trends in the movement activity over time. The classification unit 110 can hence process structured representations of the movement activity derived from the spatial representations of anatomical landmarks, together with optionally integrated sensor-derived inputs, to generate classifications or inferred characteristics of the movement activity suitable for downstream analysis, monitoring, or clinical assessment.

In some embodiments, the classification unit 110 can be configured to generate classification outputs corresponding to one or more predefined or learned categories based on analysis of movement activity of the infant. For example, the classification unit 110 can classify the movement activity into one or more categories, including, without limitation, categories corresponding to types of movement activity such as gross motor activity, fine motor activity, voluntary movement, involuntary movement, repetitive behaviors, or other movement-related classifications. The classification unit 110 can further generate higher-level classification outputs or inferred indicators based on the analyzed data. The classification outputs can include, for example, indicators associated with neuromotor characteristics, neuromuscular development, gaze-related behavior, posture-related characteristics, or other physiological or movement-related conditions inferred from the movement activity. In some embodiments, the classification unit 110 can analyze multimodal data, including movement feature data 52 derived from spatial representations of anatomical landmarks in combination with sensor-derived data such as visual data, thermal data, inertial data, or other physiological signals, to generate such classification outputs. In this manner, the classification unit 110 can generate structured classification outputs representing categorized or inferred characteristics of movement activity derived from spatial representations of anatomical landmarks, optionally enhanced through integration with multimodal sensor data.

The classification unit 110 can further optionally generate confidence values, probability scores, or other measures of certainty associated with the classification outputs. In certain embodiments, the classification unit 110 can generate classification data 112 that includes one or more labels, scores, probability values, or structured outputs representing categorized or inferred characteristics of the infant’s movement activity or physiological condition. In this manner, the classification unit 110 can transform movement feature data 52, which can include structured representations of movement activity, and associated sensor-derived inputs into structured, interpretable categorical representations that facilitate downstream processing, including comparative analysis, trend detection, and model-based assessment of infant movement activity and physiological characteristics.

In certain embodiments, the one or more machine learning models employed by the classification unit 110 can be trained using multimodal, time-synchronized training data that includes combinations of sensor-derived data, pose data, and movement feature data corresponding to known or labeled conditions, movement categories, or physiological states. The training data can include, for example, video data, image data, pose-based representations, movement feature data, and, in some embodiments, physiological measurements such as heart rate, respiratory rate, or oxygen saturation, as well as other sensor-derived signals.

The training process can involve supervised learning, semi-supervised learning, self-supervised learning, or other machine learning techniques to learn relationships between input data and corresponding classification outputs. In certain embodiments, the models can be trained to identify patterns in movement and multimodal data that correlate with specific movement classifications or physiological indicators. The training can further include temporal modeling of sequential data to capture dynamic changes in movement over time. In some embodiments, the trained models can be updated, refined, or adapted based on additional data, including new sensor data collected during operation of the system 100, annotated datasets, or domain-specific training data. The classification unit 110 can therefore utilize models that are pre-trained, fine-tuned, or otherwise adapted for specific populations, environments, or clinical use cases. The classification unit 110 can generate classification data 112 that can be transmitted to one or more downstream system for further analysis, aggregation, or presentation. In this manner, the classification unit 110 provides a classification layer that operates on movement feature data and associated inputs to generate structured, interpretable categorizations suitable for monitoring, assessment, and further processing within the system 100.

The detection and assessment system 100 can include an optional health scoring unit 120 configured to receive the classification data 112 and, in certain embodiments, one or more portions of the movement feature data 52, normalized pose data 42 representing spatial representations of anatomical landmarks, and/or preprocessed sensor data 22, and to generate one or more health-related scores or assessment outputs representing characteristics of the infant. For example, the health scoring unit 120 can be configured to aggregate, weight, and analyze the classification outputs in the classification data 112, which can be derived from movement activity of the infant, together with associated data, to generate quantitative or semi-quantitative scores corresponding to one or more characteristics of the movement activity or physiological condition of the infant. The scores can include, for example, movement activity scores, variability scores, asymmetry scores, activity-level scores, or other composite indicators derived from spatial representations of anatomical landmarks and associated sensor-derived inputs.

In certain embodiments, the scores can represent aggregated or composite measures of movement activity derived from temporal changes in spatial representations of anatomical landmarks, including measures associated with discrete movement events, continuous movement states, or temporal movement patterns. The health scoring unit 120 can employ one or more computational models, statistical techniques, rule-based systems, or combinations thereof to determine such scores. In this manner, the health scoring unit 120 can transform structured representations of movement activity and associated classification outputs, derived from spatial representations of anatomical landmarks, into higher-level quantitative indicators suitable for downstream analysis, monitoring, or clinical assessment.

The health scoring unit 120 can also be configured generate one or more composite scores based on a combination of multiple classification outputs, temporal trends, and movement feature data 52 derived from spatial representations of anatomical landmarks. For example, the health scoring unit 120 can combine classification outputs corresponding to movement activity of the infant, including discrete movement events, continuous movement states, and temporal movement patterns, together with temporal characteristics to generate aggregated indicators representing overall movement activity or changes in movement activity over time. In certain embodiments, the health scoring unit 120 can apply weighting factors, thresholds, or normalization techniques to ensure that the generated scores are consistent across different infants, time periods, or data conditions. The health scoring unit 120 can be further configured to analyze temporal changes in the classification data 112, which can be derived from movement activity based on temporal changes in spatial representations of anatomical landmarks, and associated inputs to generate trend-based scores or indicators. This analysis can include identifying increases or decreases in specific categories of movement activity, detecting deviations from baseline movement activity, or determining temporal patterns over defined time intervals.

In some embodiments, the health scoring unit 120 can generate confidence values, probability measures, or uncertainty indicators associated with the generated scores. The health scoring unit 120 can further generate structured output data, referred to herein as health score data 122, which can include one or more scores, indices, trend indicators, confidence measures, or associated metadata representing characteristics of the infant’s movement activity or physiological condition. In certain embodiments, the health score data 122 can include composite indices representing variability, asymmetry, or coordination of movement activity across one or more anatomical regions. In this manner, the health scoring unit 120 can transform classification outputs and structured representations of movement activity, derived from spatial representations of anatomical landmarks, into composite, temporally aware indicators suitable for downstream analysis, monitoring, or clinical assessment.

The health scoring unit 120 can employ one or more trained models to generate the health-related scores. The models can be trained using multimodal, time-synchronized training data that includes movement feature data 52 derived from spatial representations of anatomical landmarks, classification data 112, pose data representing spatial representations of anatomical landmarks, and/or sensor-derived data corresponding to known or labeled conditions or outcomes. The training process can include supervised learning, semi-supervised learning, or other machine learning techniques to learn relationships between input data and corresponding score outputs associated with movement activity or physiological condition of the infant. In some embodiments, the models can be configured to learn weighting relationships, feature importance, or temporal dependencies associated with movement activity derived from temporal changes in spatial representations of anatomical landmarks that contribute to the generated scores. The health scoring unit 120 can be further configured to update or adapt scoring models over time based on additional data, including newly collected sensor data, annotated datasets, or domain-specific training data. In this manner, the health scoring unit 120 can employ models that are pre-trained, fine-tuned, or otherwise adapted for specific populations, environments, or monitoring conditions.

The optional health scoring unit 120 can generate health score data 122, which can include one or more scores, indices, or composite indicators representing characteristics of movement activity or physiological condition of the infant, including composite indices representing variability, asymmetry, or coordination of movement activity across one or more anatomical regions. In certain embodiments, the health score data 122 can further include risk scores or predictive indicators associated with developmental or physiological conditions of the infant. The generated scores can be used to support monitoring, comparison across time, detection of temporal trends, or identification of changes in movement activity or physiological characteristics of the infant. The health scoring unit 120 can transform classification data 112 and associated inputs, derived from spatial representations of anatomical landmarks and related sensor-derived data, into structured, quantitative or semi-quantitative assessment outputs that facilitate downstream interpretation, monitoring, and analysis of infant movement activity and physiological characteristics.

The detection and assessment system 100 can also include the display unit 90 configured to receive and present one or more outputs generated by upstream components of the system, including, for example, the classification data 112 and the health score data 122. In certain embodiments, the display unit 90 can operate in a manner consistent with that described above with respect to FIG. 1, including generating one or more user interfaces for presenting information in a human-readable and clinically interpretable format. The display unit 90 can be configured to present classification outputs and associated information derived from movement activity of the infant, including classifications corresponding to discrete movement events, continuous movement states, or temporal movement patterns derived from temporal changes in spatial representations of anatomical landmarks. The display unit 90 can further present one or more health-related scores, indices, or composite indicators generated by the health scoring unit 120, including scores associated with movement activity, variability, asymmetry, coordination, or physiological condition of the infant.

The display unit 90 can present the information using one or more visualization formats, including, for example, graphical representations, charts, time-series plots, heatmaps, trajectory visualizations of anatomical landmarks, skeletal overlays, dashboards, or other user interface elements that facilitate interpretation of movement activity and physiological characteristics of the infant. In certain embodiments, the display unit 90 can present temporal trends associated with the classification data 112 and/or health score data 122, including changes over time, comparisons to prior data, baseline deviations, or aggregated representations of movement activity across one or more anatomical regions. The display unit 90 can also present confidence values, probability scores, or uncertainty indicators associated with the displayed data.

In some embodiments, the display unit 90 can be configured to generate one or more alerts 92 based on the received data. The alerts 92 can be generated when one or more predefined conditions, thresholds, or criteria are satisfied, such as when a health-related score exceeds or falls below a specified range, when a change in classification data or movement activity is detected over time, or when a combination of classification outputs and health score data indicates a potential condition of interest. In certain embodiments, the alerts 92 can be associated with severity levels, priority indicators, or classifications, and can be configured to prompt further evaluation, including clinical or monitoring evaluation. The display unit 90 can further be configured to communicate the alerts 92 and/or associated data to one or more external systems or devices, including computing devices, mobile devices, monitoring systems, electronic health record systems, or systems within a healthcare facility, thereby enabling remote monitoring or review by a clinical care provider.

In certain embodiments, the display unit 90 can operate in conjunction with an artificial intelligence assistant configured to receive the classification data 112, health score data 122, and/or alerts 92 and to generate contextualized outputs based thereon. The artificial intelligence assistant can be configured to generate natural language summaries, explanations of movement activity, descriptions of temporal trends, or responses to user queries associated with the infant’s movement activity or physiological data. In some embodiments, the artificial intelligence assistant can further generate recommendations, notifications, risk indicators, or suggested actions based on the received data and predefined criteria, and can present such outputs via one or more user interfaces, including graphical or conversational interfaces. In this manner, the artificial intelligence assistant can enhance interpretability and usability of system outputs by converting structured representations of movement activity and associated data into user-consumable and context-aware information. As such, the display unit 90 can transform structured classification outputs and health score data, derived from spatial representations of anatomical landmarks and associated sensor-derived inputs, into interpretable visualizations, alerts, and contextualized outputs that support monitoring, assessment, and evaluation of infant movement activity and physiological characteristics.

The illustrated detection and assessment system 100 can be configured to generate a plurality of indicators, classifications, and/or scores associated with movement activity and physiological characteristics of the infant based on outputs of the pose estimation unit 30, movement extraction unit 50, classification unit 110, and optional health scoring unit 120. The generated outputs can be derived from spatial representations of anatomical landmarks and associated sensor-derived data, and can represent structured, interpretable characterizations of the infant’s movement activity over time. For example, the detection and assessment system 100 can generate one or more neuromotor scores derived from movement features associated with movement activity, including frequency, amplitude, coordination, and temporal characteristics derived from temporal changes in spatial representations of anatomical landmarks, as represented in the movement feature data 52 and classification data 112. The scores can provide quantitative or semi-quantitative representations of movement activity and can be tracked over time to identify temporal trends, changes, or deviations from baseline movement activity.

In certain embodiments, the detection and assessment system 100 can further generate indicators associated with neuromuscular development based on observed movement activity, inter-limb coordination, variability, asymmetry, and temporal consistency of movement activity. Additionally, the system 100 can generate posture-related indicators, including representations associated with spinal alignment or curvature, based on spatial relationships among anatomical landmarks determined from the pose data 32 and normalized pose data 42. The detection and assessment system 100 can also generate indicators associated with anti-gravity movement based on limb elevation, movement amplitude, and posture-related features derived from spatial representations of the anatomical landmarks, optionally in combination with additional sensor-derived inputs such as inertial or pressure data. The detection and assessment system 100 can further generate indicators associated with gaze-related behavior based on gaze-related data, including eye position, head orientation, and visual attention characteristics, as determined from the preprocessed sensor data 22 and associated analyses.

Further, the detection and assessment system 100 can be configured to identify movement activity patterns associated with neurological events based on detection of repetitive, involuntary, or otherwise atypical movement activity patterns in the movement feature data 52 and classification data 112. The system 100 can analyze the movement activity patterns over time to generate indicators associated with neurological activity and, in some embodiments, to support generation of alerts 92 when one or more predefined conditions or thresholds are satisfied. In certain embodiments, such indicators can include risk scores or predictive indicators associated with neurological, developmental, or physiological conditions. The detection and assessment system 100 can thus transform multimodal sensor data into structured indicators, classifications, and scores representing movement activity and physiological characteristics of the infant, including composite indices associated with variability, asymmetry, or coordination of movement activity across one or more anatomical regions.

In operation, the detection and assessment system 100 can receive the sensor data 16 from one or more sensors 14 configured to capture information associated with the infant, including, for example, image data, video data, inertial data, gaze-related data, thermal data, and/or other physiological or environmental data. The sensor data 16 can be transmitted to the data acquisition unit 20, which can generate preprocessed sensor data 22 by performing one or more preprocessing operations, including conditioning, synchronization, filtering, and formatting, and, in certain embodiments, generating temporally aligned data representations suitable for downstream computational processing.

The preprocessed sensor data 22 can be conveyed to the pose estimation unit 30, which can employ one or more computational models, including machine learning models, to determine pose data 32 representing spatial representations of anatomical landmarks of the infant over time. For example, the pose estimation unit 30 can utilize computer vision models, keypoint detection models, transformer-based models, sequence models, or multimodal models to identify and track the anatomical landmarks and to generate spatial and temporal representations of body segments and posture. The pose data 32 can then be transmitted to the data cleaning and normalization unit 40, which can generate normalized pose data 42 by refining, standardizing, and temporally aligning the spatial representations of the anatomical landmarks. In certain embodiments, the data cleaning and normalization unit 40 can further generate model-ready data representations, including normalized feature vectors, temporally segmented data windows, or encoded data structures suitable for input into downstream computational models.

The normalized pose data 42 can be received by the movement extraction unit 50, which can analyze the spatial representations of the anatomical landmarks over time to identify and characterize movement activity of the infant. As described herein, the movement activity can include discrete movement events, continuous movement states, and temporal movement patterns derived from temporal changes in spatial representations of anatomical landmarks. In certain embodiments, the movement extraction unit 50 can employ one or more computational techniques, including statistical methods, signal processing techniques, and, in some embodiments, machine learning models, to derive movement features and temporal characteristics of the movement activity. The movement extraction unit 50 can generate movement feature data 52 having structured representations of movement activity, including trajectories of anatomical landmarks, kinematic features, and associated temporal characteristics.

The movement feature data 52 can be conveyed to the classification unit 110, which can employ one or more computational models, including for example machine learning models, to classify the movement activity of the infant. In certain embodiments, the classification unit 110 can analyze sequences of movement feature data 52, and optionally additional data derived from the normalized pose data 42 and/or preprocessed sensor data 22, to generate classification data 112 comprising categorized or classified representations of movement activity, including categories corresponding to discrete movement events, continuous movement states, and temporal movement patterns. The classification unit 110 can assign one or more category labels and can associate the labels with confidence values, probability scores, or ranking information. The classification unit 110 can perform the categorization or classification based on individual movement events, sequences of movement events, or aggregated temporal windows, and can assign categories or classes corresponding to movement activity types, movement states, movement patterns, voluntary or involuntary movement, symmetry or asymmetry of movement activity, posture-related characteristics, gaze-related behavior, or other movement activity or physiological characteristics. The classification unit 110 can further generate composite or hierarchical classifications based on combinations of the categories and can associate such classifications with temporal context.

The classification data 112 generated by the classification unit 110 can be optionally transmitted to the health scoring unit 120, which can generate health score data 122 by aggregating and analyzing classification outputs and associated data derived from spatial representations of anatomical landmarks. The health scoring unit 120 can employ one or more computational models, including statistical models, rule-based systems, and/or machine learning models, to generate one or more scores, indices, or composite indicators representing characteristics of movement activity or physiological condition of the infant. In certain embodiments, the health score data 122 can include composite indices representing variability, asymmetry, or coordination of movement activity across one or more anatomical regions, as well as risk scores or predictive indicators associated with developmental or physiological conditions. The health scoring unit 120 can further incorporate temporal trends, weighting factors, and learned relationships in generating such scores.

The classification data 112 and/or health score data 122 can be transmitted to the display unit 90, which can generate one or more user interfaces for presenting the data in a human-readable and clinically interpretable format. The display unit 90 can present visualizations including graphical representations, time-series plots, trajectory visualizations of anatomical landmarks, dashboards, and summary indicators representing movement activity and associated scores. In certain embodiments, the display unit 90 can present temporal trends, baseline comparisons, and confidence indicators associated with the displayed data.

In some embodiments, the display unit 90 can generate one or more alerts 92 when one or more predefined conditions or thresholds are satisfied, including conditions associated with changes in movement activity, classification data, or health score data. The alerts 92 can be associated with severity levels or priority indicators and can be configured to prompt further evaluation, including clinical or monitoring evaluation. The display unit 90 can further communicate such alerts and associated data to external systems or devices to support remote monitoring or review. In certain embodiments, the system 100 can operate in conjunction with an artificial intelligence assistant configured to receive classification data 112, health score data 122, and/or alerts 92 and to generate contextualized outputs, including natural language summaries, explanations of movement activity, descriptions of temporal trends, risk indicators, or recommended actions, thereby enhancing interpretability and usability of the system outputs. The detection and assessment system 100 can hence process the multimodal sensor data 16 through a sequence of data acquisition, pose estimation, normalization, movement activity extraction, classification, and optional scoring operations to transform the sensor data into structured representations of movement activity and associated quantitative indicators, which can be presented, analyzed, and acted upon to support monitoring, trend analysis, prediction, and evaluation of infant movement activity and physiological characteristics.

The present invention is also directed to another embodiment of a detection and assessment system configured to perform multimodal analysis of sensor-derived data to assess infant health and development. In this embodiment, the system can employ a combination of processing units configured to extract one or more of movement-related data, skin-related optical feature data, and physiological data from one or more sensing modalities, and to analyze the data to identify patterns associated with selected physiological conditions. The system can further be configured to generate categorized or classified representations, optional quantitative scores, and alerts based on the analyzed data, thereby enabling non-invasive, multimodal assessment of infant condition at the point of care.

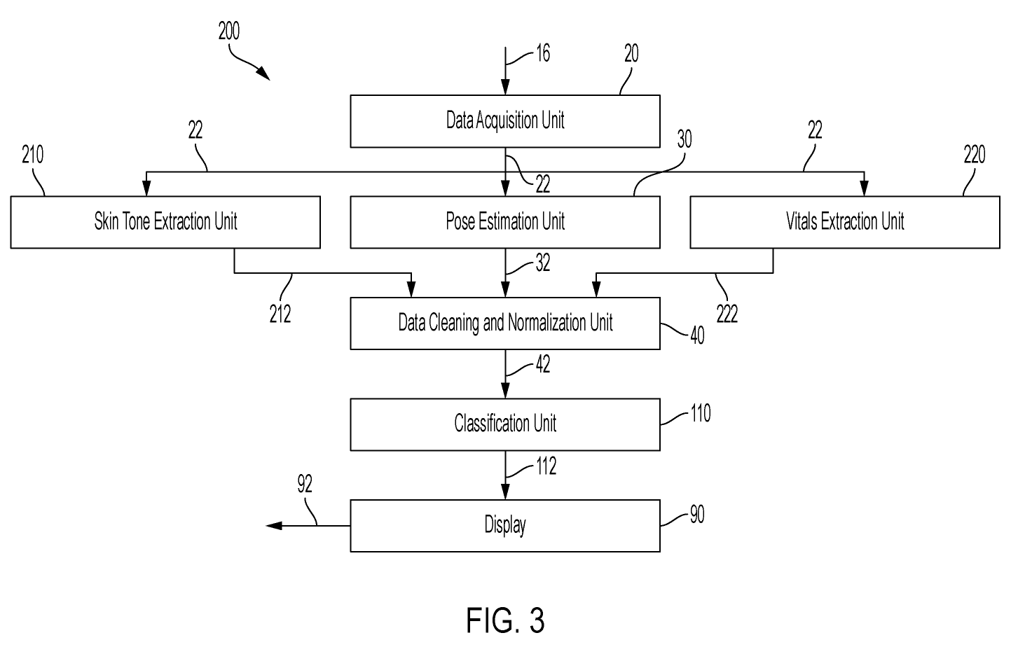

As shown in FIG. 3, the present invention is directed to yet another embodiment of a detection and assessment system 200 for assessing infant health and development at the point of care in infants, such as neonates, in a healthcare facility. The illustrated detection and assessment system 200 can employ one or more units that are similar to the units described herein in connection with the detection and assessment systems 10 and 100 of FIGS. 1 and 2, and like reference numerals indicate like parts throughout the various views. The illustrated detection and assessment system 200 can include a data acquisition unit 20, a pose estimation unit 30, a data cleaning and normalization unit 40, a classification unit 110, and a display unit 90, each of which can operate in a manner consistent with the corresponding units described above. The detection and assessment system 200 can further include one or more additional specialized processing units, including a skin tone extraction unit 210 and a vitals extraction unit 220, which can be configured to extract and analyze additional physiological characteristics in the form of physiological data associated with the infant from sensor-derived data 16. The skin tone extraction unit 210 can be configured to process image or video data to determine skin-related characteristics, including color-based features or spectral information associated with physiological conditions. The vitals extraction unit 220 can be configured to derive physiological parameters from the sensor data 16, including, for example, heart rate, respiratory rate, oxygen saturation, or other vital indicators. In certain embodiments, the vitals extraction unit 220 can derive such parameters from non-contact sensing modalities, including camera-based or thermal-based measurements, and/or from contact-based sensors where available.

With continued reference to FIG. 3, the illustrated detection and assessment system 200 captures sensor data 16 from one or more sensors 14 associated with the infant 12. The sensors 14 can include, for example, imaging sensors (e.g., RGB cameras, infrared cameras, and the like), thermal sensors, and other contact or non-contact sensing devices configured to capture visual, thermal, and/or physiological data. The sensor data 16 can include image data, video data, thermal data, and other sensor-derived data representative of the infant’s condition.

The sensor data 16 can be received by the data acquisition unit 20, which can generate preprocessed sensor data 22 by performing one or more preprocessing operations, including, for example, signal conditioning, filtering, synchronization, formatting, and temporal alignment of data received from one or more sensors. The data acquisition unit 20 can generate preprocessed sensor data 22 that can include structured or semi-structured data representations suitable for downstream processing by one or more units of the system 200. In certain embodiments, the preprocessed sensor data 22 can include temporally aligned and, optionally, multimodal data representations that facilitate subsequent determination of spatial representations of anatomical landmarks and analysis of movement activity.

The preprocessed sensor data 22 can be conveyed to the pose estimation unit 30, which can be configured to process the preprocessed sensor data 22 to determine pose data 32 representing spatial representations of anatomical landmarks of the infant over time. The pose estimation unit 30 can identify and track anatomical landmarks and generate spatial representations of body segments, posture, and orientation in two-dimensional or three-dimensional space across or over time, thereby providing a structural and spatial characterization of the infant’s body that can be used to derive movement activity based on temporal changes in the spatial representations. The pose estimation unit 30 can optionally employ one or more computational models, including machine learning models such as computer vision models, keypoint detection models, transformer-based models, or other models configured to identify and track anatomical landmarks from the preprocessed sensor data 22 and to generate the pose data 32. The pose data 32 can include for example positions, orientations, and, in some embodiments, temporal trajectories of the anatomical landmarks, which can form the basis for subsequent processing to identify and characterize the movement activity of the infant.

In addition to the processing of movement activity described above, the detection and assessment system 200 can include a skin tone extraction unit 210 configured to process image data, video data, and/or thermal data included in the preprocessed sensor data 22 to determine skin-related characteristics of the infant. In certain embodiments, the skin tone extraction unit 210 can analyze one or more regions of interest corresponding to exposed skin areas of the infant, which can be identified based on spatial representations of anatomical landmarks or body segments, and can extract one or more skin-related optical features associated with the infant’s skin. The skin-related optical features can include, for example, color-based features (e.g., red, green, and blue (RGB) channel values, chromaticity coordinates, hue, and saturation), intensity-based features (e.g., luminance, brightness, or grayscale intensity), and spectral-based features (e.g., wavelength-dependent reflectance or absorption characteristics). In certain embodiments, the skin-related optical features can further include normalized or derived representations, such as color ratios, reflectance characteristics, spatial distributions of optical properties, and temporal variations in such features over time. In some embodiments, the skin-related optical features can be determined within one or more color spaces, including for example RGB, hue saturation value (HSV), Luma, Blue-difference Chroma, Red-difference Chroma (YCbCr), or other color representations. In certain embodiments, the skin tone extraction unit 210 can generate structured representations of the skin-related characteristics, which can be temporally aligned with the spatial representations of anatomical landmarks and associated movement activity for downstream analysis by one or more units of the system 200.

According to certain embodiments, the skin tone extraction unit 210 can employ one or more computational techniques, including image processing algorithms, signal processing methods, and/or machine learning models, to analyze the preprocessed sensor data 22 and to determine skin-related characteristics of the infant. For example, the skin tone extraction unit 210 can employ one or more processing techniques to identify and isolate regions of interest corresponding to exposed skin areas, which can be determined based on spatial representations of anatomical landmarks or body segments, to reduce background interference and improve accuracy of feature extraction. The skin tone extraction unit 210 can further employ processing techniques to correct for lighting conditions, normalize color representations, and quantify color-based, intensity-based, or spectral-based features associated with the infant’s skin. In certain embodiments, the skin tone extraction unit 210 can apply color normalization techniques, segmentation algorithms to isolate skin regions, and temporal analysis methods to track changes in skin-related optical features over time, including changes that can be temporally aligned with movement activity derived from spatial representations of anatomical landmarks.

The skin tone extraction unit 210 can also optionally employ one or more trained machine learning models, including convolutional neural networks, vision transformers, or other image-based models, to identify patterns in skin-related optical features or color variations that are associated with selected types of physiological conditions. The models can be configured to process any spatial and temporal variations in the skin-related optical features across one or more anatomical regions of the infant. Variations in the skin-related optical features over time or across different anatomical regions can be indicative of changes in underlying physiological states. For example, changes in color distribution, chromaticity, perfusion-related optical characteristics, or other derived optical features can be associated with physiological conditions, including infection-related states such as sepsis, particularly when considered in combination with movement activity and/or other sensor-derived data processed by the system 200.

The models employed by the skin tone extraction unit 210 can be trained using training data that can include multimodal datasets comprising visual and/or thermal representations of infant skin under a variety of conditions. The training data can include, for example, image and video data capturing infants with varying skin tones, lighting conditions, and physiological states, as well as corresponding labeled or annotated data. The annotations can include pixel-level or region-level labels identifying skin regions, as well as labels associated with skin-related characteristics, including color distributions, perfusion-related indicators, temperature variations, or other observable features. In certain embodiments, the training data can further include reference data associated with physiological conditions, such as infection-related indicators (e.g., sepsis-associated skin changes), jaundice-related color variations, or other clinically relevant patterns, where such labels are available. In some embodiments, the models can be trained using supervised learning techniques in which labeled datasets are used to learn mappings between input data and corresponding output features or classifications. In other embodiments, semi-supervised or self-supervised learning techniques can be employed to learn feature representations from large volumes of unlabeled or partially labeled data. The training process can include augmentation of the training data, such as variation in lighting conditions, color normalization, and synthetic perturbations, to improve robustness across different environments and patient populations.

The models can further include, for example, convolutional neural networks (CNNs), vision transformers, or other image-processing architectures configured to extract spatial and spectral features from the input data. In certain embodiments, temporal models or sequence-based models can be employed to analyze changes in skin-related features over time. The models can also incorporate preprocessing pipelines, including color space transformations, normalization techniques, and segmentation algorithms for isolating skin regions prior to feature extraction. In certain embodiments, the trained models can be validated using separate validation datasets that include diverse infant populations and imaging conditions to evaluate model performance and generalization. The models can be configured to generate outputs including skin tone data 212, which can include extracted features, normalized color metrics, temporal trends, and indicators associated with physiological characteristics. Further, the models employed by the skin tone extraction unit 210 can be updated or refined over time using additional training data, including data collected during operation of the system 200, annotated datasets, or domain-specific datasets. The models can therefore be pre-trained, fine-tuned, or otherwise adapted to specific environments, populations, or sensing configurations. In this manner, the skin tone extraction unit 210 can employ trained computational models to process sensor-derived data and to generate structured skin tone data 212 representing skin-related characteristics of the infant that can be used for downstream analysis, classification, and assessment.

The skin tone extraction unit 210 can be configured to generate skin tone data 212, which can include structured representations of extracted skin-related optical features derived from one or more regions of interest corresponding to anatomical regions of the infant. In certain embodiments, the regions of interest can be identified based on spatial representations of anatomical landmarks or body segments. The skin tone data 212 can include, for example, skin-related optical feature information such as color metrics, normalized color values, spatial distributions of optical characteristics across anatomical regions, temporal trends in skin tone variation, and derived indicators associated with physiological conditions.

In certain embodiments, the skin tone data 212 can include indicators associated with perfusion, oxygenation, or other physiological states reflected in skin coloration, which can be determined based on temporal and spatial variations in optical characteristics and/or thermal characteristics across one or more anatomical regions identified based on spatial representations of anatomical landmarks, where the optical characteristics can include skin-related optical characteristics corresponding to the infant’s skin. The skin-related optical characteristics can include, for example, color-based features, intensity-based features, spectral-based features, temperature-related characteristics, and derived or normalized representations thereof. The skin tone data 212 can be temporally aligned with movement activity derived from the spatial representations of anatomical landmarks to enable integrated analysis of physiological and movement-related characteristics of the infant. By way of example, the skin tone extraction unit 210 can analyze variations in skin-related optical characteristics and/or thermal characteristics, including variations in skin color, temperature, and related optical properties across one or more anatomical regions over time, to generate indicators associated with physiological conditions, including, for example, infection-related states (e.g., sepsis) and other conditions reflected in variations in skin coloration, such as jaundice, perfusion-related conditions, or other physiological states. The conditions can manifest as changes in skin perfusion, pallor, mottling, color distribution, temperature variation, or other observable characteristics derived from the skin-related optical characteristics and/or thermal characteristics. In certain embodiments, the indicators can include risk indicators or predictive indicators associated with physiological conditions, based on patterns or temporal trends in the skin-related optical characteristics and/or thermal characteristics. The skin tone data 212 can therefore provide additional physiological insight beyond movement activity-based analysis and can be used in conjunction with other data streams, including movement feature data 52, classification data 112, health score data 122, and/or other sensor-derived data, for multimodal assessment of the infant. The skin tone data 212 can be transmitted to one or more downstream units, including the classification unit 110, the health scoring unit 120, and/or the display unit 90, for further analysis, categorization, scoring, and presentation.

The skin tone extraction unit 210 can be configured to generate skin tone data 212, which can include structured representations of extracted optical characteristics and/or thermal characteristics associated with one or more anatomical regions of the infant, wherein the optical characteristics can include skin-related optical characteristics corresponding to the infant’s skin. The anatomical regions can be identified based on spatial representations of anatomical landmarks. The skin-related optical characteristics can include, for example, color-based features, intensity-based features, spectral-based features, temperature-related characteristics, and derived or normalized representations thereof. The skin tone data 212 can include, for example, skin-related optical feature information such as color metrics, normalized color values, spatial distributions of optical characteristics across anatomical regions, temporal trends in skin tone variation, and derived indicators associated with physiological conditions. In certain embodiments, the skin tone data 212 can be temporally aligned with movement activity derived from the spatial representations of anatomical landmarks. The skin tone data 212 can further include indicators associated with perfusion, oxygenation, or other physiological states reflected in skin coloration or temperature, which can be determined based on temporal and spatial variations in the skin-related optical characteristics and/or thermal characteristics.

By way of example, the skin tone extraction unit 210 can analyze temporal and spatial variations in the optical characteristics and/or thermal characteristics, including the skin-related optical characteristics such as skin color and temperature-related features, across one or more anatomical regions identified based on the spatial representations of anatomical landmarks, to generate indicators associated with physiological conditions, including infection-related states (e.g., sepsis) and other conditions reflected in variations in skin coloration or temperature, such as jaundice, perfusion-related conditions, or other physiological states. The conditions can manifest as changes in skin perfusion, pallor, mottling, color distribution, temperature variation, or other observable characteristics derived from the skin-related optical characteristics and/or thermal characteristics. The skin tone data 212 can therefore provide additional physiological insight beyond movement activity-based analysis and can be used in conjunction with other data streams, including movement feature data 52, classification data 112, health score data 122, and/or vital sign data, for multimodal assessment of the infant. In certain embodiments, the skin tone data 212 can be temporally aligned with movement activity derived from spatial representations of anatomical landmarks to enable integrated analysis. The skin tone data 212 can be transmitted to one or more downstream units, including the classification unit 110 and/or the display unit 90, for further analysis, categorization, scoring, and presentation.

In addition to the movement activity-based and skin-related processing described above, the illustrated detection and assessment system 200 can include a vitals extraction unit 220 configured to receive and process the preprocessed sensor data 22 to determine physiological parameters associated with the infant. The vitals extraction unit 220 can analyze one or more types of sensor-derived inputs from the sensors 14, including, for example, image data, video data, thermal data, inertial data, and/or signals or other data obtained from contact-based sensors, to extract physiological signal characteristics and derive vital sign-related features and corresponding physiological measurements. In certain embodiments, the vitals extraction unit 220 can analyze the inputs across one or more anatomical regions identified based on spatial representations of anatomical landmarks.

In certain embodiments, the vitals extraction unit 220 can integrate data from a plurality of sensing modalities, including contact and non-contact modalities, such as pulse oximeters, electrocardiogram (ECG) sensors, thermal imaging sensors, respiratory sensors, and other sensing devices, to derive physiological parameters, including, for example, heart rate, respiratory rate, oxygen saturation, perfusion-related indicators, and other vital sign metrics. The physiological parameters can be derived from temporal and/or spatial variations in physiological signal characteristics associated with the infant. The vitals extraction unit 220 can derive the physiological parameters using non-contact sensing techniques, such as remote photoplethysmography (rPPG) based on analysis of temporal variations in skin-related optical characteristics, thermal imaging techniques for detecting respiratory patterns or temperature-related variations, and motion-based analysis for identifying breathing cycles or other physiological rhythms. In other embodiments, the vitals extraction unit 220 can process signals received from contact-based sensors, such as pulse oximeters or electrocardiogram (ECG) sensors, where available. In certain embodiments, the extracted physiological parameters can be temporally aligned with movement activity derived from spatial representations of anatomical landmarks to enable integrated analysis of physiological and movement-related characteristics. Variations or anomalies in the physiological parameters or underlying physiological signal characteristics can be indicative of changes in underlying physiological states, including physiological instability, infection-related conditions, or other physiological conditions.

The vitals extraction unit 220 can optionally employ one or more computational techniques, including signal processing methods, statistical analysis, and/or machine learning models, to extract and refine the physiological parameters. For example, the vitals extraction unit 220 can perform filtering, frequency-domain analysis, peak detection, and temporal signal decomposition to isolate physiological signals from noise. In certain embodiments, the vitals extraction unit 220 can employ trained machine learning models, including time-series models, convolutional neural networks, or transformer-based models, to identify patterns in the sensor-derived data and to estimate physiological parameters with improved robustness and accuracy under varying conditions.

In certain embodiments, the vitals extraction unit 220 can generate vitals extraction data 222 representing structured physiological data for downstream processing. The vitals extraction unit 220 can operate in conjunction with the skin tone extraction unit 210 to provide a multimodal data stream reflecting both internal physiological characteristics and external observable characteristics of the infant. Such multimodal data can be processed by downstream units, including the classification unit 110, to identify co-occurring patterns across physiological data and skin-related optical features that are associated with physiological conditions, including infection-related states such as sepsis.

The one or more computational models employed by the vitals extraction unit 220 can be trained using training data that includes sensor-derived data and corresponding reference physiological measurements. The sensor-derived data can include, without limitation, image data, video data, thermal data, optical data, inertial data, and/or signals from contact-based sensors. The corresponding reference physiological measurements can include, for example, heart rate, respiratory rate, oxygen saturation, perfusion-related values, temperature-related values, or other vital sign measurements obtained from reference monitoring equipment or annotated clinical data. The training data can include time-synchronized data in which video frames, thermal frames, motion signals, or other sensor signals are temporally aligned with measured physiological parameters. The models can be trained to learn relationships between subtle variations in the sensor-derived data and corresponding physiological parameters. For example, a model can be trained to associate periodic color changes in skin regions with heart rate, motion of the chest or abdomen with respiratory rate, thermal patterns with temperature-related indicators, or optical signal variations with perfusion or oxygenation-related indicators.

The training process can include supervised learning, semi-supervised learning, self-supervised learning, transfer learning, or combinations thereof. In certain embodiments, the model can be trained to generate estimated vital sign values, confidence values, signal quality indicators, or trend information based on received sensor data. The training process can also include preprocessing, filtering, temporal segmentation, normalization, and data augmentation to improve robustness across different infants, lighting conditions, sensor positions, motion artifacts, and clinical environments. In some embodiments, the models can be validated using separate validation datasets containing sensor-derived data and reference vital sign measurements. The models can be pre-trained, fine-tuned, or adapted based on infant-specific data, neonatal population data, facility-specific data, or additional operational data collected during use of the system 200.

In certain embodiments, the vitals extraction unit 220 can analyze temporal variations and correlations across multiple data streams included in the preprocessed sensor data 22 to improve reliability of extracted physiological parameters, where the analysis can be performed based on physiological signal characteristics derived from optical characteristics and/or thermal characteristics, motion-related characteristics associated with movement activity, and/or signals obtained from contact-based sensors, and, in certain embodiments, across one or more anatomical regions identified based on spatial representations of anatomical landmarks. In certain implementations, the optical characteristics can include skin-related optical characteristics that are extracted and structured by the skin tone extraction unit 210 and provided as input to the vitals extraction unit 220. For example, the vitals extraction unit 220 can correlate motion-related characteristics, thermal characteristics, and optical characteristics, including skin-related optical characteristics provided by the skin tone extraction unit 210, to distinguish physiological signals from artifacts caused by movement activity or environmental conditions.

The vitals extraction unit 220 can be configured to generate vitals extraction data 222, which can include structured representations of extracted physiological data, including physiological signal characteristics, derived physiological features, and physiological parameters. The vitals extraction data 222 can include, for example, estimated physiological parameter values (e.g., heart rate, respiratory rate, oxygen saturation), temporal trends, signal quality indicators, confidence values, uncertainty measures, and derived indicators associated with the infant’s physiological condition. In certain embodiments, the vitals extraction data 222 can represent direct physiological measurements and/or estimates derived from sensor data, including non-contact sensing techniques such as remote photoplethysmography (rPPG) based on analysis of temporal variations in skin-related optical characteristics, including those provided by the skin tone extraction unit 210, as well as thermal imaging techniques and motion-based analysis. The vitals extraction data 222 can be temporally aligned with movement activity derived from spatial representations of anatomical landmarks and can be transmitted to one or more downstream units, including the classification unit 110 and/or the display unit 90 for further analysis, categorization, scoring, and presentation. In certain embodiments, the vitals extraction data 222 can be used in combination with movement feature data 52 and skin tone data 212 to support multimodal assessment of infant health and development.

With further reference to FIG. 3, the illustrated detection and assessment system 200 can include a data cleaning and normalization unit 40 configured to receive and process multiple types of data generated by the upstream units, including the pose data 32 generated by the pose estimation unit 30, the skin tone data 212 generated by the skin tone extraction unit 210, and the vitals extraction data 222 generated by the vitals extraction unit 220. The data cleaning and normalization unit 40 can be configured to refine, standardize, and prepare the data for downstream processing by one or more units of the system 200. In certain embodiments, the data cleaning and normalization unit 40 can apply one or more data conditioning, cleaning, and normalization operations to the received data, including filtering, noise reduction, artifact removal, outlier detection, interpolation or imputation of missing data, temporal smoothing, and cross-modal alignment of data streams. For example, the data cleaning and normalization unit 40 can remove spurious pose points in the pose data 32, normalize spatial representations of anatomical landmarks across time or reference frames, correct for lighting or environmental variations in the skin tone data 212, and filter noise or motion-related artifacts from the vitals extraction data 222. In certain embodiments, the data cleaning and normalization unit 40 can further standardize data formats, coordinate systems, feature representations, and temporal synchronization across the pose data 32, skin tone data 212, and vitals extraction data 222 to generate normalized data representations suitable for downstream processing. The data cleaning and normalization unit 40 can thus improve the reliability, consistency, and interoperability of the data across different sensing modalities.

The data cleaning and normalization unit 40 can further be configured to normalize the received data into a common representation or format. In certain embodiments, this can include normalization of spatial coordinates in the pose data 32 to a reference frame, normalization of skin-related optical feature values in the skin tone data 212 to account for lighting or sensor variability, and normalization of physiological data in the vitals extraction data 222 to standardized units or scales. The data cleaning and normalization unit 40 can also perform temporal alignment of the data, such that the pose data 32, the skin tone data 212, and the vitals extraction data 222 are synchronized across a common time axis.

The data cleaning and normalization unit 40 can also optionally generate integrated or aligned multimodal data representations by associating corresponding elements of one or more of the pose data 32, the skin tone data 212, and the vitals extraction data 222 based on temporal and/or spatial relationships. For example, the data cleaning and normalization unit 40 can associate optical features, such as skin-related optical features, with corresponding anatomical regions identified in the pose data 32 and correlate the features with contemporaneous physiological data in the vitals extraction data 222. The data cleaning and normalization unit 40 can be further configured to generate normalized data 42, which can include structured, temporally aligned, and standardized representations of the multimodal data suitable for downstream processing. The normalized data 42 can include normalized pose representations, normalized skin-related optical feature data, normalized physiological data, and/or combined feature vectors representing integrated multimodal information comprising combinations of the input data types.

Further, the data cleaning and normalization unit 40 can also be configured to prepare model-ready data representations, including feature vectors, temporally segmented data windows, or encoded data structures, for input into downstream computational models, including those employed by the classification unit 110. In this manner, the data cleaning and normalization unit 40 can transform heterogeneous, sensor-derived data into consistent and structured data representations that facilitate reliable analysis, classification, and assessment of infant health and development.

With continued reference to FIG. 3, the detection and assessment system 200 can include a classification unit 110 configured to receive and process one or more types of data generated by the upstream system units, including, for example, normalized pose data 42 representing spatial representations of anatomical landmarks derived from the pose data 32 generated by the pose estimation unit 30, the skin tone data 212 generated by the skin tone extraction unit 210, the vitals extraction data 222 generated by the vitals extraction unit 220, and any combinations thereof. The classification unit 110 can be configured to analyze the received data to generate classification data 112 including one or more categorized or classified representations associated with the infant’s movement activity, physiological condition, and/or observable characteristics. In certain embodiments, the classification unit 110 can perform multimodal analysis by correlating features derived from the normalized pose data 42, the normalized skin tone data 212, and the normalized vitals extraction data 222 to identify patterns, relationships, or conditions associated with the infant.

In certain embodiments, the classification unit 110 can be configured to perform categorization or classification of the normalized pose data 42 representing spatial representations of anatomical landmarks to generate movement activity-related classifications. The classifications can include, for example, movement activity type categories (e.g., gross motor movement, fine motor movement, limb-specific movement, whole-body movement), movement activity state categories (e.g., active, inactive, resting, transitioning), movement activity pattern categories (e.g., repetitive motion, irregular motion, coordinated motion, uncoordinated motion), posture-related categories (e.g., supine, prone, side-lying, extended, flexed), and symmetry-related categories (e.g., symmetric movement activity, asymmetric movement activity), and subcategories or combinations thereof. In certain embodiments, the classification unit 110 can further generate hierarchical or multi-label classifications representing relationships among the categories and/or combinations of movement activity characteristics over time.

The classification unit 110 can be configured to process the normalized version of the skin tone data 212, including skin-related optical characteristic information derived from optical characteristics and/or thermal characteristics, to generate skin-related classifications. The classifications can include, for example, categories or classes associated with color distribution (e.g., uniform coloration, non-uniform coloration), perfusion-related categories (e.g., normal perfusion, reduced perfusion), temperature-related or thermal variation categories, and other categories indicative of physiological conditions reflected in skin characteristics, including observable features such as pallor, mottling, or color variation patterns, and subcategories or combinations thereof. In certain embodiments, such classifications can be generated based on temporal and/or spatial variations in the skin-related optical characteristics across one or more anatomical regions.

The classification unit 110 can also be configured to process the normalized version of the vitals extraction data 222, which can include physiological signal characteristics, derived physiological features, and physiological parameters, to generate physiological classifications. The classifications can include, for example, categories associated with heart rate ranges (e.g., within an expected range, elevated, reduced), respiratory patterns (e.g., regular, irregular), oxygenation-related categories, perfusion-related categories, and other categories associated with physiological states or trends, and subcategories or combinations thereof. In certain embodiments, the classifications can be based on temporal trends, correlations, or deviations from baseline associated with the physiological parameters.

The classification unit 110 can also be configured to perform cross-categorization of data derived from multiple modalities by analyzing relationships and correlations among the pose data 32, skin tone data 212, and vitals extraction data 222. The classification unit 110 can generate multimodal categories by combining movement activity-related classifications with skin-related classifications and physiological classifications to produce composite or hierarchical category representations. The cross-categorization can include identification of patterns in which movement characteristics, skin-related optical features, and physiological parameters collectively correspond to a particular state, condition, or trend.

In certain embodiments, the classification unit 110 can be configured to analyze the multimodal data to identify patterns associated with selected physiological conditions. For example, the classification unit 110 can detect co-occurring patterns including variations in physiological data (e.g., heart rate, respiratory rate, or perfusion-related indicators), changes in skin-related optical features (e.g., pallor, mottling, or color variation), and/or changes in movement characteristics (e.g., reduced activity or altered movement patterns). The combined patterns can be categorized as being associated with selected types of physiological instability or infection-related conditions, including sepsis, and can be used to generate classification outputs, indicators, or alerts for further clinical evaluation.

The classification unit 110 can employ one or more computational techniques, including rule-based logic, statistical models, and/or machine learning models, to perform the classification and cross-categorization processes. The models can include, without limitation, time-series models, convolutional neural networks, transformer-based models, or multimodal models configured to process heterogeneous data inputs. The classification unit 110 can analyze data over one or more temporal windows and can assign one or more category labels to a given time interval or sequence, optionally with associated confidence values or probability measures. The classification unit 110 can generate classification data 112 including one or more category labels, confidence values, temporal associations, and/or composite or hierarchical classifications representing multimodal interpretations of the infant’s condition. The classification unit 110 can thus transform the multimodal sensor-derived data, including the movement-related data, the skin-related optical feature data, and the physiological data, into structured, categorized representations that facilitate integrated assessment of infant health and development.

In certain embodiments, the detection and assessment system 200 can optionally include a health scoring unit 120, similar to the one employed in FIG. 2, that can be configured to receive and process the classification data 112 generated by the classification unit 110, and, in some embodiments, one or more underlying data types including the normalized pose data representing spatial representations of anatomical landmarks, the normalized skin tone data, and the normalized vitals extraction data. The health scoring unit 120 can be configured to generate one or more scores, indices, or other quantitative representations associated with the infant’s movement activity, physiological condition, and/or multimodal patterns identified across the processed data. The health scoring unit 120 can generate movement activity-related scores based on movement classifications derived from the normalized pose data 42. The scores can include, for example, activity level scores, movement variability scores, symmetry or asymmetry indices, coordination scores, and temporal stability scores. The scores can represent quantitative measures of movement activity over one or more time intervals and can be used to assess temporal trends, changes, or deviations in motor behavior.

The health scoring unit 120 can also be configured to generate physiological scores based on the vitals extraction data 222, which can include physiological signal characteristics, derived physiological features, and physiological parameters. The scores can include, for example, indices associated with heart rate variability, respiratory regularity, oxygenation-related measures, perfusion-related indicators, and other physiological trends. In certain embodiments, the scores can reflect deviations from expected ranges, temporal fluctuations, baseline deviations, or stability characteristics of the physiological parameters.

In other embodiments, the health scoring unit 120 can generate skin-related scores based on the skin tone data 212, including skin-related optical characteristic information derived from optical characteristics and/or thermal characteristics. The scores can include, for example, indices associated with color uniformity, perfusion-related characteristics, thermal variation, or temporal changes in skin-related optical characteristics. These scores can represent quantitative measures of observable skin characteristics and their variation over time and across one or more anatomical regions.

The health scoring unit 120 can further be configured to generate composite or multimodal scores by integrating any combination of movement activity-related scores, physiological scores, and skin-related scores. The composite scores can represent aggregated or weighted combinations of multiple data sources and can be configured to reflect overall patterns associated with the infant’s condition. For example, the health scoring unit 120 can generate a composite score representing a level of physiological stability, a risk-related score associated with multimodal deviations, or a trend-based score reflecting changes in the infant’s condition over time.

The health scoring unit 120 can employ one or more computational techniques to generate the scores, including, without limitation, rule-based algorithms, statistical models, weighted aggregation methods, and/or machine learning models. In certain embodiments, the scoring can be based on predefined thresholds, learned relationships from training data, temporal trend analysis, or combinations thereof. The health scoring unit 120 can analyze data over one or more time windows and can generate scores that are updated continuously or periodically to reflect real-time or near real-time changes in the underlying data. The generated scores can be associated with confidence values, uncertainty measures, or signal quality indicators. The scores can signify relative levels, trends, or deviations associated with the infant’s movement, physiological state, or multimodal condition, and can be used to support monitoring, assessment, or alert generation. For example, the scores can indicate increasing or decreasing levels of activity, stability, or variability, or can reflect patterns associated with physiological instability or conditions such as infection-related states. The health scoring unit 120 can generate scoring data 122 including one or more scores, indices, temporal trends, and associated metadata. The scoring data 122 can be transmitted to one or more downstream units, including the display unit 90, for presentation, visualization, alert generation, and/or further analysis. In this manner, the health scoring unit 120 can transform categorized multimodal data into structured quantitative representations that facilitate monitoring, trend analysis, and assessment of infant health and development.

With continued reference to FIG. 3, the detection and assessment system 200 can include a display unit 90 configured to receive and present one or more types of data generated by upstream units, including, for example, the classification data 112 generated by the classification unit 110, the vitals extraction data 222 generated by the vitals extraction unit 220, the skin tone data 212 generated by the skin tone extraction unit 210, the pose data 32 generated by the pose estimation unit 30, and, in an optional embodiments, the scoring data 122 generated by the optional health scoring unit 120. The display unit 90 can be configured to generate one or more user interfaces for presenting such data in a human-readable format.

The display unit 90 can present movement activity-related classifications derived from the pose data 32, including, for example, movement types, movement states, posture classifications, and symmetry-related classifications. The display unit 90 can further present physiological data and associated classifications derived from the vitals extraction data 222, including, without limitation, heart rate, respiratory rate, oxygenation-related indicators, and temporal trends associated with such physiological parameters. The display unit 90 can also present skin-related optical feature information and associated classifications derived from the skin tone data 212. The visualizations can include representations of color variation, perfusion-related indicators, thermal distributions, and temporal changes in skin-related optical features. In certain embodiments, the display unit 90 can present region-based or spatial visualizations indicating variations across different portions of the infant’s body. The display unit 90 can also present multimodal or composite information, including integrated visualizations that combine movement-related data, physiological data, and skin-related optical feature data. The visualizations can include dashboards, charts, graphs, heatmaps, trajectory plots, and time-series representations that illustrate trends, correlations, and patterns across the multiple data modalities.

In embodiments in which the health scoring unit 120 is employed, the display unit 90 can present one or more scores or indices, including movement-related scores, physiological scores, skin-related scores, and composite scores, along with associated confidence values or trend indicators. The display unit 90 can present such scores in a manner that facilitates interpretation, including relative comparisons, threshold-based indicators, or temporal progression.

The display unit 90 can further be configured to generate one or more alerts 92 based on the data received from upstream units. In certain embodiments, the alerts 92 can be generated when one or more predefined conditions, thresholds, or patterns are satisfied. For example, the alerts 92 can be generated based on changes in physiological data (e.g., heart rate or respiratory rate outside of expected ranges), changes in skin-related optical features (e.g., patterns indicative of perfusion changes or temperature variation), changes in movement characteristics (e.g., reduced activity or abnormal movement patterns), and/or combinations thereof. The alerts 92 can also be generated based on multimodal pattern detection, including patterns identified by the classification unit 110 that are associated with physiological instability or infection-related conditions, including sepsis. Such alerts can represent indicators that one or more data streams exhibit co-occurring deviations or patterns warranting further evaluation.

The alerts 92 can be presented visually, audibly, or via other notification mechanisms, including graphical indicators, color-coded signals, messages, or alarms. In certain embodiments, the alerts 92 can be prioritized, categorized, or associated with severity levels, and can include contextual information, such as contributing data sources, temporal trends, or associated confidence values. The display unit 90 can further be configured to update the presented data and alerts in real time or near real time, thereby enabling continuous monitoring and assessment of the infant’s condition. In this manner, the display unit 90 and alerts 92 can facilitate timely interpretation of multimodal data and support informed decision-making by clinical personnel.

In certain embodiments, the detection and assessment system 200 can be configured to identify patterns associated with selected types of infection-related conditions, including sepsis, based on multimodal data processing. For example, the classification unit 110 can analyze any combination of the pose data 32, skin tone data 212, and vitals extraction data 222 to detect co-occurring patterns, including variations in physiological data (e.g., heart rate, respiratory rate, or perfusion-related indicators), changes in skin-related optical features (e.g., pallor, mottling, or color variation), and changes in movement characteristics (e.g., reduced activity or altered movement patterns). The classification unit 110 can categorize the combined patterns as being associated with physiological instability or infection-related conditions. In embodiments in which the optional health scoring unit 120 is employed, the health scoring unit 120 can generate one or more scores or indices reflecting the degree, trend, or likelihood associated with such multimodal patterns, thereby providing a quantitative representation of the identified condition-associated patterns. The classification data 112 and/or scoring data 122 can be conveyed to the display unit 90, which can present the associated classifications, scores, and trends, and can generate one or more alerts 92 when predefined conditions, thresholds, or pattern combinations are satisfied. Such alerts 92 can indicate the presence of multimodal patterns associated with sepsis or other infection-related states and can prompt further clinical evaluation.

In certain embodiments, the detection and assessment system 200 can identify patterns associated with the infection-related conditions, including sepsis, based on an analysis of the multimodal data. The physiological data included in the vitals extraction data 222 can provide primary indicators of physiological instability, including variations in heart rate, respiratory rate, perfusion-related parameters, or other vital signs. In addition, the system 200 can employ supplemental data sources, including skin tone data 212 and/or the pose data 32, to enhance detection and interpretation of the patterns. For example, skin-related optical feature information can provide indicators associated with changes in perfusion or oxygenation, while movement-related data can provide context associated with activity levels or behavioral changes. The classification unit 110 can integrate these data types to identify co-occurring patterns associated with physiological conditions, and the optional health scoring unit 120 can generate scores reflecting the degree or trend of such patterns. In this manner, the system 200 can leverage both primary physiological data and supplemental sensor-derived indicators to improve robustness and reliability of condition-associated pattern detection.

In operation, and with reference to FIG. 3, the detection and assessment system 200 can capture sensor data 16 associated with an infant using one or more sensors 14, including, for example, imaging sensors, thermal sensors, and contact-based or non-contact sensing devices. The sensor data 16 can include image data, video data, thermal data, and/or other sensor-derived data representative of the infant’s condition. The sensor data 16 can be received by the data acquisition unit 20, which can preprocess the data to generate preprocessed sensor data 22, including performing operations such as filtering, synchronization, formatting, and temporal alignment. The preprocessed sensor data 22 can be conveyed to one or more downstream system units, including the pose estimation unit 30, the skin tone extraction unit 210, and the vitals extraction unit 220. The pose estimation unit 30 can process the preprocessed sensor data 22 to generate pose data 32 representing spatial representations of anatomical landmarks of the infant over time, which can be used to derive movement activity based on temporal changes in the spatial representations. The skin tone extraction unit 210 can process the preprocessed sensor data 22 to extract optical characteristics and/or thermal characteristics, including skin-related optical characteristics, and generate skin tone data 212 representing observable characteristics such as color variation, chromaticity, perfusion-related indicators, and temperature-related variations. The vitals extraction unit 220 can process the preprocessed sensor data 22, and in certain embodiments skin-related optical characteristics provided by the skin tone extraction unit 210, to derive physiological signal characteristics and generate vitals extraction data 222 representing one or more physiological parameters, including heart rate, respiratory rate, oxygenation, and other physiological indicators.

The pose data 32, skin tone data 212, and vitals extraction data 222 can be received by the data cleaning and normalization unit 40, which can perform data conditioning, cleaning, and normalization operations, including noise reduction, artifact removal, outlier detection, interpolation or imputation of missing data, normalization, and temporal alignment, to generate normalized data 42. The normalized data 42 can include structured, temporally aligned, and cross-modally consistent representations of the multimodal data suitable for downstream processing.

The classification unit 110 can receive and process one or more of the normalized data 42 associated with the skin tone data 212, the vitals extraction data 222, and/or the pose data 32 to perform multimodal analysis. In particular, the classification unit 110 can analyze the multimodal data to identify co-occurring patterns, including variations in physiological parameters (e.g., heart rate, respiratory rate, or perfusion-related indicators), changes in skin-related optical characteristics (e.g., pallor, mottling, or color variation), and changes in movement activity (e.g., reduced activity, altered movement patterns, or changes in coordination). The classification unit 110 can categorize such combined patterns as being associated with physiological instability or physiological conditions, including infection-related states (e.g., sepsis), and can generate classification data 112 representing such categorizations.

In certain embodiments, the detection and assessment system 200 can optionally employ a health scoring unit 120, which can receive the classification data 112 and/or the underlying multimodal data to generate one or more scores or indices representing the degree, trend, or likelihood associated with the identified patterns. The scoring data 122 can include quantitative representations of physiological stability, risk-related indicators, or temporal trends associated with the infant’s condition. The classification data 112 and/or the scoring data 122 can be conveyed to the display unit 90, which can generate one or more user interfaces for presenting the data in a human-readable format, including visualizations of physiological parameters, skin-related optical characteristics, movement activity, and associated temporal trends. The display unit 90 can further generate one or more alerts 92 when predefined conditions, thresholds, or multimodal pattern combinations are satisfied. Such alerts 92 can indicate the presence of multimodal patterns associated with physiological conditions, including infection-related states such as sepsis, and can prompt further clinical evaluation. In this manner, the detection and assessment system 200 can transform sensor-derived data into structured multimodal representations, classifications, and optional quantitative scores, thereby enabling identification of patterns associated with physiological conditions, including sepsis, and supporting real-time or near real-time assessment of infant health.

According to another embodiment of the present invention, the detection and assessment system 200 can be configured to identify patterns associated with jaundice based on an analysis of multimodal data. The multimodal data can refer to data derived from multiple sensing modalities and/or data types, including movement-related data, skin-related optical feature data, and physiological data, which may be obtained from one or more sensors. In the current embodiment, the multimodal data at least includes the skin tone data 212 generated by the skin tone extraction unit 210. The skin tone extraction unit 210 can process image data, video data, and/or optical sensor data to extract skin-related optical features associated with the infant’s skin. The skin-related optical features can include color-based features (e.g., red, green, and blue channel values), chromaticity information, hue and saturation characteristics, and normalized color metrics that can be indicative of changes in skin coloration. The skin tone extraction unit 210 can analyze one or more regions of interest corresponding to exposed skin areas, including, for example, facial regions, torso regions, extremities, or other anatomical areas, to detect variations in skin coloration over time. The extracted skin-related optical features can be processed to identify patterns associated with yellowing of the skin, which may be indicative of elevated bilirubin levels. For example, the detection and assessment system 200 can detect shifts in color distributions, changes in chromaticity coordinates, or variations in color channel ratios that are associated with jaundice-related skin coloration.

In certain embodiments, the skin tone extraction unit 210 can track the skin-related optical features over time and can compare such features across successive time intervals or relative to prior measurements to identify temporal trends or progressive changes in skin coloration. Such temporal comparisons can be used to generate indicators associated with changes in skin coloration that are associated with jaundice-related patterns.

The classification unit 110 can receive the skin tone data 212 indicative of the skin-related optical features and can categorize the extracted features into one or more skin-related classifications, including categories associated with normal coloration and categories associated with color variation patterns indicative of jaundice. The classification unit 110 can further analyze temporal changes in the skin-related optical features to identify progressive or persistent color shifts over time. In certain embodiments, the detection and assessment system 200 can optionally employ multimodal analysis by combining the skin tone data 212 with other data types, including pose data 32 and vitals extraction data 222, to improve robustness and reduce false positives. For example, the system 200 can correlate skin-related optical features with physiological data or movement data to account for lighting changes, motion artifacts, or environmental conditions.

In embodiments where the optional health scoring unit 120 is employed, the health scoring unit 120 can generate one or more scores or indices associated with the degree or progression of the jaundice-associated patterns based on the classification data 112. The scores can represent, for example, relative levels of color deviation, temporal trends in skin coloration, or composite indicators derived from multiple regions of interest.

The classification data 112 and/or the scoring data 122 can be conveyed to the display unit 90, which can present the associated skin-related classifications, scores, and trends. The display unit 90 can further generate one or more alerts 92 when predefined conditions or thresholds associated with jaundice-related patterns are satisfied. Such alerts can indicate the presence of patterns associated with jaundice and can prompt further clinical evaluation. In this manner, the detection and assessment system 200 can transform sensor-derived image data into structured skin-related optical feature data and categorized representations that facilitate identification of patterns associated with jaundice in a non-invasive manner.

It is to be understood that although the present invention has been described above in terms of particular embodiments, the foregoing embodiments are provided as being illustrative only and are not intended to limit or define the scope of the invention. Various other embodiments, including but not limited to those described herein are also within the scope of the claims and current invention. For example, the foregoing elements, units, modules, tools, engines, comparators, models, and components described herein may be further divided into additional components or sub-components or units or joined together to form fewer components for performing the same functions.

Any of the functions disclosed herein may be implemented using means for performing those functions. Such means include, but are not limited to, any of the components or units disclosed herein, as well as known electronic and computing devices and associated components.

The techniques described herein may be implemented, for example, in hardware, one or more computer programs tangibly stored on one or more computer-readable media, firmware, hardware or any combination thereof. The techniques described herein may be implemented in one or more computer programs executing on (or executable by) a programmable computer or electronic device having any combination of any number of the following: a processor, a storage medium readable and/or writable by the processor (including, for example, volatile and non-volatile memory and/or storage elements), memory, an input device, an output device, and a display. Program code may be applied to input entered using the input device to perform the functions described and to generate output using the output device. The units and subsystems of the systems 10 and 140 can be implemented by suitable electronic devices.

The term computing device or electronic device as used herein can refer to any device, such as a computer, smart phone, server and the like, that includes a processor and a non-transitory computer-readable memory or storage capable of storing computer-readable instructions, and in which the processor is capable of executing the computer-readable instructions in the memory. The terms electronic device, computer, computer device and system and computing device or system refer herein to a system containing one or more computing or electronic devices that are configured to implement one of more units, modules, or components of the systems 10 and 140 of the present invention.

Embodiments of the present invention include features which are only possible and/or feasible to implement with the use of one or more computers or servers, processors, and/or other elements of a computer or server system. Such features are either impossible or impractical to implement mentally and/or manually. For example, embodiments of the present invention may operate on digital electronic processes which can only be created, stored, modified, processed, and transmitted by computing devices and other electronic devices having suitable processors and memory elements. Such embodiments, therefore, address problems which are inherently computer-related and solve such problems using computer technology in ways which cannot be solved manually or mentally by humans.

Any claims herein which by implication or affirmatively require an electronic device such as a computer or server, a processor, a memory, storage, or similar computer-related elements, are intended to require such elements, and should not be interpreted as if such elements are not present in or required by such claims. Such claims are not intended, and should not be interpreted, to cover methods and/or systems which lack the recited computer-related elements. For example, any method claims herein which recite that the claimed method is performed or implemented by a computer, a processor, a memory, and/or similar computer-related element, is intended to, and should only be interpreted to, encompass methods which are performed by the electronic device or computer-related element(s). Such a method claim should not be interpreted, for example, to encompass a method that is performed mentally or by hand (e.g., using pencil and paper). Similarly, any product or computer readable medium claim herein which recites that the claimed product includes a computer, a processor, a memory, and/or similar computer-related element, is intended to, and should only be interpreted to, encompass products which include the computer-related element(s). Such a product claim should not be interpreted, for example, to encompass a product that does not include computer-related element(s).

Embodiments of the present invention solve one or more problems that are inherently rooted in computer technology. For example, embodiments of the present invention solve the problem of how to effectively detect, monitor and assess movement activity in an infant so as to make a physiological determination based on the sensed data. There is no analog to this problem in the non-computer environment, nor is there an analog to the solutions disclosed herein in the non-computer environment.

Furthermore, embodiments of the present invention represent improvements to computer and communication technology itself. For example, the systems 10, 100 and 200 of the present invention can optionally employ a specially programmed or special purpose computer in an improved computer system, which may, for example, be implemented within a single computing device.

Each computer program within the scope of the claims below may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a non-transitory machine-readable storage or memory device for execution by a computer processor. Method steps of the invention may be performed by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays). A computer can generally also receive (read) programs and data from, and write (store) programs and data to, a non-transitory computer-readable storage medium such as an internal disk (not shown) or a removable disk. These elements can also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium.

Any data disclosed herein may be implemented, for example, in one or more data structures tangibly stored on a non-transitory computer-readable medium. Embodiments of the invention may store such data in such data structure(s) and read such data from such data structure(s).

It should be appreciated that various concepts, systems and methods described above can be implemented in any number of ways, as the disclosed concepts are not limited to any particular manner of implementation or system configuration. Examples of specific implementations and applications that are discussed herein are primarily for illustrative purposes and for providing or describing the operating environment of the system of the present invention. The system 10 and/or elements or units thereof can employ one or more electronic or computing devices, such as one or more servers, clients, computers, laptops, smartphones and the like, that are networked together, or which are arranged so as to effectively communicate with each other. The network can be any type or form of network. The devices can be on the same network or on different networks. In some embodiments, the network system may include multiple, logically grouped servers. In one of these embodiments, the logical group of servers may be referred to as a server farm or a machine farm. In another of these embodiments, the servers may be geographically dispersed. The electronic devices can communicate through wired connections or through wireless connections. The clients can also be generally referred to as local machines, clients, client nodes, client machines, client computers, client devices, endpoints, or endpoint nodes. The servers can also be referred to herein as servers, server nodes, or remote machines. In some embodiments, a client has the capacity to function as both a client or client node seeking access to resources provided by a server or server node and as a server providing access to hosted resources for other clients. The clients can be any suitable electronic or computing device, including for example, a computer, a server, a smartphone, a smart electronic pad, a portable computer, and the like. The systems 10, 100, and 200 or any associated units or components of the system can employ one or more of the illustrated computing devices and can form a computing system. Further, the server may be a file server, application server, web server, proxy server, appliance, network appliance, gateway, gateway server, virtualization server, deployment server, SSL VPN server, or firewall, or any other suitable electronic or computing device, such as the electronic device. In one embodiment, the server may be referred to as a remote machine or a node. In another embodiment, a plurality of nodes may be in the path between any two communicating servers or clients.

We claim:

1. A detection and assessment system for assessing movement activity of an infant, comprising:
one or more sensors configured to generate sensor data associated with the infant;
a data acquisition unit configured to receive the sensor data and to generate processed sensor data by performing one or more preprocessing operations on the sensor data;
a pose estimation unit configured to process the processed sensor data to determine spatial representations of anatomical landmarks of the infant over time and to generate pose data representing the spatial representations of the anatomical landmarks over time;
a data cleaning and normalization unit configured to process the pose data by applying one or more data cleaning and normalization operations and to generate normalized pose data representing the spatial representations of the anatomical landmarks over time; and
a movement extraction unit configured to process the normalized pose data representing the spatial representations of the anatomical landmarks over time to identify and characterize the movement activity based on temporal changes in the spatial representations, and to generate movement feature data having structured representations of the movement activity.

2. The system of claim 1, further comprising at least one comparator unit configured to receive the movement feature data and to generate comparison data associated with the movement activity, the comparison data including at least one of limb movement comparison data representing intra-limb movement activity and inter-limb comparison data representing movement activity across contralateral anatomical regions.

3. The system of claim 2, further comprising a movement variance quantification unit configured to receive at least one of the limb movement comparison data or the inter-limb comparison data and to generate movement variability data representing variability or asymmetry of the movement activity.

4. The system of claim 3, further comprising a display unit configured to receive at least one of the movement feature data and the comparison data and to display representations of the movement activity, including at least one of movement feature representations, comparative representations, or the movement variability data to the user.

5. The system of claim 1, further comprising:
a limb movement comparator unit configured to receive and process the movement feature data and to perform a temporal comparison of movement activity of a selected limb based on the movement feature data, and to generate limb movement comparison data including structured representations of temporal changes in the movement activity for the selected limb;
a cross-midline movement comparator unit configured to receive and process the movement feature data and to perform a comparison of movement activity between contralateral anatomical regions of the infant derived from the spatial representations of the anatomical landmarks based on the movement feature data, and to generate inter-limb comparison data including structured representations of comparative relationships between the movement activity of the contralateral anatomical regions; and
a movement variance quantification unit configured to (i) receive the limb movement comparison data to generate movement variability data representing variability of the movement activity of the selected limb, (ii) receive the inter-limb comparison data to generate movement variability data representing asymmetry or coordination of the movement activity across the contralateral anatomical regions, or (iii) receive both the limb movement comparison data and the inter-limb comparison data to generate movement variability data representing integrated variability and asymmetry of the movement activity, including relationships between variability of the selected limb and asymmetry across the contralateral anatomical regions.

6. The system of claim 5, further comprising a display unit configured to receive at least one of the limb movement comparison data or the inter-limb comparison data and to present representations of the movement activity, including representations of temporal changes in movement activity for the selected limb and representations of comparative relationships between movement activity of the contralateral anatomical regions.

7. The system of claim 5, wherein the limb movement comparator unit is configured to perform a temporal comparison of movement activity of a selected limb based on the movement feature data across temporal intervals to detect changes, trends, or deviations in the movement activity, and wherein the cross-midline movement comparator unit is configured to perform a comparison of movement activity between contralateral anatomical regions of the infant based on the movement feature data and to generate inter-limb comparison data comprising structured representations of comparative relationships between the movement activity of the contralateral anatomical regions, including relative differences, timing relationships, or coordination.

8. The system of claim 3, wherein the movement variance quantification unit is configured to process (i) the limb movement comparison data independently to generate intra-limb variability metrics, (ii) the inter-limb comparison data independently to generate asymmetry metrics associated with contralateral anatomical regions, or (iii) both the limb movement comparison data and the inter-limb comparison data to generate integrated variability and asymmetry metrics.

9. The system of claim 1, wherein the movement feature data includes trajectories of anatomical landmarks and associated movement features including at least one of displacement, velocity, acceleration, frequency, duration, or range of motion.

10. The system of claim 1, wherein the movement activity comprises at least one of discrete movement events, continuous movement states, or temporal movement patterns characterizing the movement of the infant over time.

11. The system of claim 6, wherein the display unit is configured to generate one or more alerts based on one or more predefined conditions associated with at least one of the movement activity, variability of the movement activity, or asymmetry of the movement activity.

12. The system of claim 5, wherein the movement variance quantification unit is configured to determine temporal trends in variability or asymmetry of the movement activity over one or more time intervals.

13. The system of claim 5, wherein the limb movement comparator unit is configured to perform the temporal comparison across one or more temporal windows including sliding or overlapping intervals.

14. The system of claim 5, wherein the movement extraction unit is configured to identify and classify the movement activity into at least one of discrete movement events, continuous movement states, or temporal movement patterns.

15. The system of claim 5, wherein the movement variance quantification unit is configured to determine relationships between intra-limb variability and inter-limb asymmetry of the movement activity.

16. The system of claim 11, wherein the alerts are configured to prompt further evaluation.

17. A computer-implemented method for assessing movement activity of an infant, comprising:
receiving sensor data associated with the infant from one or more sensors;
generating processed sensor data by performing one or more preprocessing operations on the sensor data;
processing the processed sensor data to determine spatial representations of anatomical landmarks of the infant over time and generating pose data representing the spatial representations of the anatomical landmarks over time;
processing the pose data by applying one or more data cleaning and normalization operations to generate normalized pose data representing the spatial representations of the anatomical landmarks over time; and
processing the normalized pose data representing the spatial representations of the anatomical landmarks over time to identify and characterize movement activity based on temporal changes in the spatial representations, and generating movement feature data comprising structured representations of the movement activity.

18. The method of claim 17, further comprising receiving the movement feature data and generating comparison data associated with the movement activity, the comparison data including at least one of (i) limb movement comparison data representing intra-limb movement activity or (ii) inter-limb comparison data representing movement activity across contralateral anatomical regions.

19. The method of claim 18, further comprising receiving at least one of the limb movement comparison data or the inter-limb comparison data and generating movement variability data representing variability or asymmetry of the movement activity.

20. The method of claim 19, further comprising presenting representations of the movement activity, including at least one of movement feature representations, comparative representations, or movement variability data.

21. The method of claim 17, further comprising:
performing a temporal comparison of movement activity of a selected limb based on the movement feature data and generating limb movement comparison data comprising structured representations of temporal changes in the movement activity for the selected limb;
performing a comparison of movement activity between contralateral anatomical regions of the infant derived from the spatial representations of the anatomical landmarks based on the movement feature data and generating inter-limb comparison data including structured representations of comparative relationships between the movement activity of the contralateral anatomical regions; and
(i) processing the limb movement comparison data to generate movement variability data representing variability of the movement activity of the selected limb,
(ii) processing the inter-limb comparison data to generate movement variability data representing asymmetry or coordination of the movement activity across the contralateral anatomical regions, or
(iii) processing both the limb movement comparison data and the inter-limb comparison data to generate movement variability data representing integrated variability and asymmetry of the movement activity, including relationships between variability of the selected limb and asymmetry across the contralateral anatomical regions.

22. The method of claim 21, further comprising:
performing the temporal comparison across one or more temporal intervals to detect changes, trends, or deviations in the movement activity, and
generating inter-limb comparison data including relative differences, timing relationships, or coordination between the contralateral anatomical regions.

23. The method of claim 19, further comprising:
(i) processing the limb movement comparison data independently to generate intra-limb variability metrics,
(ii) processing the inter-limb comparison data independently to generate asymmetry metrics associated with contralateral anatomical regions, or
(iii) processing both the limb movement comparison data and the inter-limb comparison data to generate integrated variability and asymmetry metrics.

24. The method of claim 17, wherein the movement feature data comprises trajectories of the anatomical landmarks over time and one or more associated movement features including at least one of displacement, velocity, acceleration, frequency, duration, or range of motion.

25. The method of claim 17, wherein the movement activity comprises at least one of discrete movement events, continuous movement states, or temporal movement patterns characterizing movement of the infant over time.

26. The method of claim 20, further comprising generating one or more alerts based on one or more predefined conditions associated with at least one of the movement activity, variability of the movement activity, or asymmetry of the movement activity.

27. The method of claim 21, further comprising determining temporal trends in variability or asymmetry of the movement activity over one or more time intervals.

28. The method of claim 21, further comprising identifying and classifying the movement activity into at least one of discrete movement events, continuous movement states, or temporal movement patterns.

29. The method of claim 21, further comprising determining relationships between intra-limb variability and inter-limb asymmetry of the movement activity.

30. The method of claim 26, further comprising generating the one or more alerts to prompt further evaluation.
